(19) United States
(12) Patent Application Publication
(10) Pub. No.: US 2025/0002592 A1
(43) Pub. Date: Jan. 2, 2025

(54) METHOD FOR TREATMENT OF MODERATE TO SEVERE ATOPIC DERMATITIS

(71) Applicant: Aslan Pharmaceuticals Pte Ltd, Singapore (SG)

(72) Inventors: Alison Ward, Singapore (SG); Ferda Cevikbas, Palo Alto, CA (US); Karen Veverka, Kansas City, KS (US)

(21) Appl. No.: 18/695,502

(22) PCT Filed: Sep. 27, 2022
(86) PCT No.: PCT/SG2022/050694

§ 371 Date: Mar. 26, 2024

Related U.S. Application Data
(63) Continuation-in-part of application No. 17/929,824, filed on Sep. 6, 2022.
(63) Continuation-in-part of application No. PCT/SG2022/050102, filed on Mar. 1, 2022.
(63) Continuation-in-part of application No. 17/929,874, filed on Sep. 6, 2022.

(30) Foreign Application Priority Data
Sep. 27, 2021 (SG) 10202110688T
Sep. 27, 2021 (SG) 10202110690S
Sep. 27, 2021 (SG) 10202110730R
Oct. 29, 2021 (SG) 10202112085U
Feb. 22, 2022 (SG) 10202201722U
May 18, 2022 (SG) 10202205222Q
Aug. 26, 2022 (SG) 10202250837V
Sep. 6, 2022 (SG) 10202250923N

(51) Int. Cl. C07K 16/28; A61K 39/00; A61P 17/00

(52) U.S. Cl. CPC C07K 16/2866; A61P 17/00; A61K 2039/545

(57) ABSTRACT

The present disclosure relates to use of an anti-IL-13Rα1 antibody (such as eblasakimab) or a binding fragment thereof and pharmaceutical formulations comprising same to treat patients with atopic dermatitis (such as lesional atopic dermatitis) including a patient population that received prior treatment with dupilumab, for example to stimulate disease modification.

The present disclosure relates to use of an anti-IL-13Rα1 antibody or a binding fragment thereof and pharmaceutical formulations comprising same to treat patients with atopic dermatitis (such as lesional atopic dermatitis) including a patient population that received prior treatment with dupilumab, for example to stimulate disease modification.

### BACKGROUND

One possible way to inhibit the activity of IL-13 is to interfere with the binding of IL-13 to its receptor IL-13R, for example by using an antibody specific to IL-13R, such as an antibody specific to IL-13Rα1. An effective antibody antagonist to IL-13Rα1 may also interfere with the binding of IL-13and prevent heterodimerization of IL-4Rα and IL-13Rα1. Such an antibody could inhibit signaling of both IL-13 and IL-4 through the type II receptor while sparing IL-4 signalling through the type I receptor. Signalling through the type I receptor is essential in the induction phase of the immune response during which Th2 cells differentiate. T cells do not express IL-13Rα1 so the type II receptor plays no role in Th2 differentiation. Hence, an IL-13Rα1 antibody should not affect the overall Th1/Th2 balance. Signalling through the type II IL-4IL-13 receptor is critical during the effector-A-stage of the immune response during established allergic inflammation. Thus, blockade of the type II receptor should have a beneficial effect on many of the symptoms of allergic type disease, such as asthma, atopic dermatitis and other IL-13R-mediated conditions and should, therefore, be an effective disease modifying agent.

Antibodies against IL-13Rα1 (both monoclonal and polyclonal) have been described in the art; see, eg, WO 97/15663, WO 03/80675; WO 03/46009; WO 06/072564; Gauchat et al, 1998 Eur. J. Immunol. 28:4286-4298; Gauchat et al, 2000 Eur. J. Immunol. 30:3157-3164; Clement et al, 1997Cytokine 9(11):959 (Meeting Abstract); Ogata et al, 1998 J. Biol. Chem. 273:9864-9871; Graber et al, 1998 Eur. J. Immunol. 28:4286-4298; C. Vermot-Desroches et al, 2000 Tissue Antigens 5 (Supp. 1): 52-53 (Meeting Abstract); Poudrier et al, 2000 Eur. J. Immunol. 30:3157-3164; Akaiwa et al, 2001Cytokine 13:75-84; Cancino-Diaz et al, 2002 J. Invest. Dermatol. 119:1114-1120; and Krause et al, 2006 Mol. Immunol. 43:1799-1807.

One particularly promising anti-IL-13Rα1 antibody is described in WO2008/060813 as antibody 10G5-6. 10G5-6 as an IgG4 with a hinge stabilising serine to proline mutation (S241P Kabat numbering) is known as Eblasakimab.

Eblasakimab has been shown to bind to human IL-13Rα1 with a high affinity (for example Kd may be 500 pM). EBLASAKIMAB was shown to effectively antagonise IL-13 function through inhibiting the binding of IL-13 to its receptor IL-13Rα1 and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

Atopic dermatitis can be a very painful, demoralising and psychologically damaging disease. One method of assessing the disease is the EASI score. The score is in the range 0-72. In some instances, moderate to severe forms of the disease are not adequately controlled by topical medicines. In addition, it is not advisable for some patients to take the available topical medicines. Dupixent (dupilumab) is an antibody inhibitor of the interleukin-4 receptor alpha (IL-4Rα), which is licensed for the treatment of atopic dermatitis.

In some instances patients prescribed dupilumab stop treatment, for one or more reasons, such as adverse side effects, inadequate control of symptoms and/or problems with dosing. This leaves some patients with in moderate to severe topic dermatitis in position where they don't have suitable treatment.

Moderate atopic dermatitis generally has a score in the range 7.1 to 21.0.

Severe atopic dermatitis has scope of 21.1 or above. Sometimes severe atopic dermatitis is divided into severe and very severe disease and these groups have the range 21.1 to 50 and 50.1 to 72.0 respectively.

The inventors have performed clinical trials and have a established that whilst a whole range of atopic dermatitis patients may benefit from the treatment with an antibody or antigen binding fragment thereof, which is an inhibitor of signalling through of the IL-13Rα1 by binding the said receptor, it seems that those with a baseline EASI score of at least 16 may get the most benefit from the treatment and/or patients with lesional atopic derma.

The present invention provides a new patient population, for example patients that have had prior treatment with dupilumab and/or patients with lesional atopic dermatitis. In some instances, it is useful to combine the baseline EASI score is combined with one or more other markers, such as baseline IgE, TARC and STAT6 to define the patient population. Surprisingly these patients show the greatest improvements with treatment according to the present disclosure. In some embodiments the treatment is disease modifying. Normal levels of TARC in healthy individuals is usually up to 450 pg/ml.

Interestingly the present inventors have also established that the IL-13Ralpha-1 is upregulated in lesional atopic dermatitis so patients with lesional atopic dermatitis may especially benefit from treatment with an anti-IL-13 antibody, such as eblasakimab (for example as an independent aspect or as an embodiment of moderate/sever atopic dermatitis).

Lesional and non-lesional atopic dermatitis are different sub-groups of patients. Non-lesional AD has immune abnormalities including expansion of T-cells. It has a variable immune phenotype, which is largely determined by disease extent and severity. There is decreased hydration and impaired synthesis of lipids have been found in non-lesional atopic skin in comparison to normal skin. There is also abnormal epidermal proliferation in non-lesional atopic dermatitis with increased expression of the proliferation marker (keratin 16), in association with altered expression of terminal differentiation proteins, including loricrin (LOR), involucrin (IVL), and FLG. Additionally, an increased infiltrate of inflammatory T-cells has been demonstrated in non-lesional atopic dermatitis skin compared to skin from healthy volunteers. In one embodiment non-lesional atopic dermatitis is characterised by the absence of lesions.

Lesional skin from chronic AD patients has broad alterations in expression of terminal differentiation genes that form the cornified envelope and epidermal differentiation complex along with many differences in highly inflammatory genes in lesional AD skin. In the simplest form lesional AD is characterised by lesions.

### SUMMARY OF THE DISCLOSURE

1. An antibody or antigen binding fragment thereof, which is an inhibitor of signalling through of the IL-13 Rα1 by binding the said receptor, for use in the treatment of moderate, severe or very severe atopic dermatitis (in particular poorly controlled moderate to severe atopic dermatitis) by parenteral administration of a treatment cycle comprising a dose in the range 200 mg to 600 mg, (such as 400 to 600 mg), wherein the disease baseline is characterised by a EASI score of 16 or above (such as 17, 18, 19, 20, 21, 22, 23, 24, 25, 26, 27, 28, 29, 30, 31, 32,33, 34, 35, 36, 37, 38, 39, 40, 41, 42, 43, 44, 45, 46, 47, 48, 49, 50, 51, 52, 53, 54, 55, 56, 57, 58,59, 60, 61, 62, 63, 64, 65, 66, 67, 68, 69, 70, 71, 72).
1A A method of treatment by parenteral administration of a treatment cycle comprising a dose in the range 200 mg to 600 mg, (such as 400 to 600 mg) of an antibody or antigen binding fragment thereof, which is an inhibitor of signalling through of the IL-13 Rα1 by binding the said receptor, to a patient with moderate atopic dermatitis, severe or very severe atopic dermatitis wherein the disease baseline is characterised by a FASI score of 16 or above (such as 17, 18, 19, 20, 21, 22, 23, 24, 25, 26, 27, 28, 29, 30, 31, 32, 33, 34, 35, 36, 37, 38, 39, 40, 41,42, 43, 44, 45, 46, 47, 48, 49, 50, 51, 52, 53, 54, 55, 56, 57, 58, 59, 60, 61, 62, 63, 64, 65, 66, 67,68, 69, 70, 71, 72).
1B An antibody or antigen binding fragment thereof, which is an inhibitor of signalling through of the IL-13 Rα1 by binding the said receptor, for use in the manufacture of a medicament for the treatment of moderate, severe or very severe atopic dermatitis (in particular poorly controlled moderate to severe atopic dermatitis) by parenteral administration of a treatment cycle comprising a dose in the range 200 mg to 600 mg, (such as 400 to 600 mg), wherein the disease baseline is characterised by a EASI score of 16 or above (such as 17, 18, 19, 20, 21,22, 23, 24, 25, 26, 27, 28, 29, 30, 31, 32, 33, 34, 35, 36, 37, 38, 39, 40, 41, 42, 43, 44, 45, 46, 47,48, 49, 50, 51, 52, 53, 54, 55, 56, 57, 58, 59, 60, 61, 62, 63, 64, 65, 66, 67, 68, 69, 70, 71, 72).
1C. An antibody or antigen binding fragment thereof, which is an inhibitor of signalling through of the IL-13 Rα1 by binding the said receptor, for use in the treatment lesional atopic dermatitis (in particular poorly controlled moderate to severe atopic dermatitis) by parenteral administration of a treatment cycle comprising a dose in the range 200 mg to 600 mg, (such as 400 to 600 mg).
2. The antibody or antigen binding fragment thereof, method or use according to paragraph 1, 1A or 1B, wherein the atopic dermatitis is lesional.
3. The antibody or antigen binding fragment thereof, method or use according to any preceding paragraph, wherein the patient has previously been treated with dupilumab.
4. The antibody or antigen binding fragment thereof, method or use according to any preceding paragraph, wherein the baseline TARC level is at least 701 pg/ml, for example in the range 800 to 52,000 pg/ml (such as 800, 900, 1,000, 1500, 2,000, 2,500, 3,000, 3,500, 4,000, 4,500,5,000, 5,500, 6,000, 6,500, 7,000, 7,500, 8,000, 8,500, 9,000, 9,500, 10,000, 11,000, 12,000,13,000, 14,000, 15,000, 16,000, 17,000, 18,000, 19,000, 20,000, 21,000, 22,000, 23,000,24,000, 25,000, 26,000, 27,000, 28,000, 29,000, 30,000, 31,000, 32,000, 33,000, 34,000,35,000, 36,000, 37,000, 38,000, 39,000, 40,000, 41,000, 42,000, 43,000, 44,000, 45,000,46,000, 47,000, 48,000, 49,000, 50,000, 51,000 or 52,000 pg/ml), in particular at least 1,115 pg/ml, for example 1,115 pg/ml to 4,300 pg/ml or above 4,300 pg/ml.
5. The antibody or antigen binding fragment thereof, method or use according to any preceding paragraphs, wherein the baseline STAT6 is above normal.
6. The antibody or antigen binding fragment thereof, method or use according to any preceding paragraphs, wherein the patient has a baseline IGA score of 3 or more, which is moderate to very severe atopic dermatitis, for example 3 (moderate atopic dermatitis), 4 (severe atopic dermatitis) or 5 (very severe atopic dermatitis).
7. The antibody or antigen binding fragment thereof, method or use according to any preceding paragraph, wherein the baseline IgE levels (for example have been established/measured) are at a level of at least 130 kU/L for example at 150 kU/L such as 150 to 20,000, more specifically 130, 150, 200, 300, 400, 500, 600, 700, 750, 800, 850, 900, 950, 1000, 1500,2,000, 2,500, 3,000, 3500, 4,000, 4,500, 5,000, 5,500, 6,000, 6,500, 7,000, 7,500, 8,000, 8,500,9,000, 9,500, 10,000, 11,000, 12,000, 13,000, 14,000, 15,000, 16,000, 17,000, 18,000, 19,000,20,000 kU/L, in particular at 10,000 KU/L +/−2,000.
8. The antibody or antigen binding fragment thereof, method or use according to any preceding paragraphs, wherein the one or more the parameters (for example 2 or more or all of the parameters) have been measured prior to dosing initiation, in particular the patients has been identified as the relevant/suitable population prior to treatment.
9. The antibody or antigen binding fragment, method or use according to any preceding paragraph, wherein the patient has been identified as having one or more (for example all of the parameters) prior to starting treatment i.e. in particular the patient has been confirmed as in the population of moderate to severe dermatitis based on data other than solely clinical observation and/or EASI score.
10. The antibody or antigen binding fragment thereof, method or use according to any preceding paragraph, wherein the atopic dermatitis is moderate, for example with an EASI score in the range 16 to 21.0.
11. The antibody or antigen binding fragment thereof, method or use according to any preceding paragraph, wherein the atopic dermatitis is moderate and the TARC score is at least 2,056 +/−290 pg/ml.
12. The antibody or antigen binding fragment thereof, method or use according to any preceding paragraph, wherein the atopic dermatitis is moderate and the IgE score is at least 130 IU/L (such as in the range 130 to 6,800 IU/L more specifically 130 to 479 IU/L).
13. The antibody or antigen binding fragment thereof, method or use according to any preceding paragraph, wherein the atopic dermatitis is severe, for example with an FASI scope 21.1 or above, such as 21.1 to 50.0.
14. The antibody or antigen binding fragment thereof, method or use according to any preceding paragraph, wherein the atopic dermatitis is severe and the TARC score is at least 4,812 +/−490 pg/ml.
15. The antibody or antigen binding fragment thereof, method or use according to any one of paragraphs 1, 1A, 1B to 11, 13 or 14, wherein the atopic dermatitis is severe and the IgE score is at least 480 IU/L (such as in the range 480 to 15,100 IU/L).
16. The antibody or antigen binding fragment thereof, method or use according to any one of paragraphs 1, 1A, 1B to 11 or 13 to 15, wherein the atopic dermatitis is very severe, for example with a score in the range 50.1 to 72.0.
17. The antibody or antigen binding fragment thereof, method or use according to any preceding paragraph, wherein a reduction from baseline in EASI score is present after about two weeks from administration of the first dose (such as day 15), for example at least a 15% reduction.
18. The antibody or binding fragment thereof, method or use according to any preceding paragraph, wherein there is a reduction from baseline in EASI score is in the range 15 to 60% (for example at about day 15), for example 25 to 60% (such as 39 to 59% in particular 40 to 59, more specifically 47, 48, 49, 50, 51, 52, 53, 54, 55, 56, 57, 58 or 59%) especially at about day 15.
19. The antibody or binding fragment thereof, method or use according to any preceding paragraph, wherein there is a reduction from baseline in EASI score is in the range 50 to 100% (for example 55 to 97%) in particular at about day 29.
20. An antibody or antigen binding fragment thereof or use according to any preceding paragraph, wherein there is a reduction in EASI score is in the range −40 to −85% (for example at about day 29).
21. The antibody or antigen binding fragment thereof, method or use according to any preceding paragraphs, wherein a reduction in EASI score is present after about four weeks from administration of the first dose (such as day 29).
22. The antibody or antigen binding fragment thereof, method or use according to any preceding paragraph, wherein a reduction in EASI score is present after about six weeks from administration of the first dose (such as day 43).
23. The antibody or binding fragment thereof according to any preceding paragraph, wherein the reduction from baseline in EASI score is in the range 60 to 100% (for example 70 to 97%) in particular at about day 43.
24. An antibody or antigen binding fragment thereof according to any preceding paragraph, wherein the reduction in EASI score is in the range −25 to −85% (for example at about day 43or 57).
25. The antibody or antigen binding fragment thereof, method or use according to any preceding paragraph, wherein a reduction in EASI score is present after about eight weeks from administration of the first dose (such as day 57).
26. The antibody or binding fragment thereof, method or use according to any preceding paragraph, wherein there is a reduction from baseline in EASI score is in the range 65 to 100% (for example 70 to 100, such as 90 to 100, in particular 91, 92, 93, 94, 95, 96, 97, 98, 99 or 100%) more specifically at about day 57.
27. The antibody or binding fragment thereof, method or use according to any preceding paragraph, wherein 80% of the patient population has an EASI 50 score or less at about day 29 and/or day 57, in particular where the dosing of at least 350 mg (such as 400 mg) was employed.
28. The antibody or binding fragment thereof, method or use according to any preceding paragraph, wherein 90% of the patient population has an EASI 50 score or less at about day 57, in particular where dosing of at least 550 mg (such as 600 mg) was employed.
29. The antibody or antigen binding fragment thereof, method or use according to any preceding paragraph, wherein there is at least a 20% reduction from baseline in IgE by day 57, such as about 20 to 60% reduction from baseline.
30. The antibody or antigen binding fragment thereof, method or use according to any preceding paragraph, wherein there is at least a 50% reduction from baseline in TARC, for example by about day 15.
31. The antibody or antigen binding fragment thereof, method or use according to any preceding paragraph, wherein there is at least a 60% reduction in TARC from baseline, for example by about day 36.
32. The antibody or antigen binding fragment thereof, method or use according to any preceding paragraph, wherein there is at least a 70% reduction in TARC from baseline, for example by about day 50.
33. The antibody or antigen binding fragment thereof, method or use according to any preceding paragraph, wherein there is at least an 80% reduction in TARC from baseline, for example by about day 57.
34. The antibody or antigen binding fragment thereof, method or use according to any preceding paragraph, wherein the treatment is administered intravenously.
35. The antibody or antigen binding fragment thereof, method or use according to any one of claims 1, 1A, 1B, 1C to 33, wherein the treatment is administered subcutaneously.
36. The antibody or antigen binding fragment thereof, method or use according to any preceding paragraph, wherein multiple doses are administered in a treatment cycle (for example wherein the treatment cycle is 4 to 8 weeks, such as 8 weeks).
37. The antibody or binding fragment thereof, method or use according to paragraph 36,wherein multiple treatment cycles are administered, for example 2, 3, 4 or more treatment cycles are administered.
38. The antibody or binding fragment thereof, method or use according to paragraph 36 or 37,wherein following the treatment cycle or cycles and disease modification, maintenance therapy is administered, for example the same dose administered less frequently (for example monthly, such as 4 weekly, 5 weekly, 6 weekly, 7 weekly, 8 weekly), or a lower dose (such as 200 mg) administered the same frequency or less frequently (such as about weekly, two weekly, about three weekly, or about four weekly.
39. The antibody or antigen binding fragment thereof, method or use according to any preceding paragraph, wherein said antibody or binding fragment thereof is administered approximately weekly, (in particular in a treatment cycle (including a single cycle), especially a cycle of 8 weeks).
40. The antibody or antigen binding fragment thereof, method or use according to any one of paragraphs 1, 1A, 1B, 1C, to 38, wherein said antibody or binding fragment thereof is administered once approximately every two weeks, (in particular in a treatment cycle (incuding a single cycle), especially a cycle of 8 weeks).
41. The antibody or antigen binding fragment thereof, method or use according to any one of paragraphs 1, 1A, 1B, 1C to 38, wherein said antibody or binding fragment thereof is administered once approximately every three weeks, (in particular in a treatment cycle (including a single cycle), especially a cycle of 8 weeks).
42. The antibody or antigen binding fragment thereof, method or use according to any one of paragraphs 1, 1A, 1B, 1C to 38, wherein the antibody or binding fragment thereof is administered once approximately every four weeks (for example monthly), (in particular in a single treatment cycle, especially a cycle of 8 weeks).
43. The antibody or antigen binding fragment thereof, method or use according to any preceding paragraph, wherein a loading dose (for example the range 400 to 900 mg, such as 400, 500,600, 700, 800 or 900 mg) is employed before administration of further doses in the treatment cycle.
44. The antibody or antigen binding fragment thereof, method or use according to any one of paragraphs 1, 1A, 1B, 1C to 42, wherein the treatment does not comprise a loading dose.
45. An antibody or antigen binding fragment thereof, method or use according to any preceding paragraph, wherein the dose is 200 mg.
46. The antibody or binding fragment thereof, method or use according to any one of paragraphs 1, 1A, 1B, 1C to 44, wherein the dose is in the range 350 to 450 mg, such as 400 mg.
47. The antibody or binding fragment thereof, method or use according to any one of paragraphs 1, 1A, 1B, 1C to 44, wherein the dose is 600 mg.
48. The antibody or binding fragment thereof, method or use according to any preceding paragraphs, wherein the treatment cycles comprises, a first dose at 600 mg, followed by three weekly doses of 400 mg, for example wherein the treatment cycle is repeated twice i.e. two treatment cycles lasting 8 weeks (with or without the loading dose i.e. wherein the first dose it the same as other doses in the cycle), in particular: day 1 600 mg, approximately day 8 400 mg, approximately day 15 400 mg approximately day 22 400 mg, approximately day 29 600 mg, approximately day 36400 mg, approximately day 43 400 mg, and approximately day 50 400 mg are administered.
49. The antibody or binding fragment thereof, method or use according to any preceding paragraph, wherein disease modification, occurs by day 4, wherein day 1 is the first administration of the antibody or binding fragment thereof.
50. The antibody or binding fragment thereof, method or use according to any preceding paragraph, wherein the disease modification is a reduction from baseline in:
   a. EASI score, for example wherein the reduction is a percentage from base line, for example the reductions is in the range 10 to 55%; and/or
   b. TARC, for example as described elsewhere herein; and/or
   c. IgE, for example as described elsewhere herein.
51. The antibody or binding fragment thereof according to any preceding paragraph, wherein there is disease modification in the range −40 to −100% is achieved by about day 57 following first administration on day 1, for example maximum disease modification is achieved by about day 57.
52. The antibody or antigen binding fragment thereof, method or use according to any preceding paragraphs, wherein the antibody or binding fragment binds an epitope FFYQ (for example same epitope as the antibody with a VH shown in SEQ ID NO: 51 and a VL shown in SEQ ID NO: 53.
53. The antibody or antigen binding fragment thereof, method or use according to any preceding paragraphs, wherein the anti-IL-13 R antibody comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10.
54. The antibody or antigen binding fragment thereof, method or use according to any preceding paragraph, wherein the anti-IL-13 R antibody comprises a VH domain comprising an amino acid sequence shown in SEQ ID NO: 51 or a sequence at least 95% identical thereto.
55. The antibody or antigen binding fragment thereof, method or use according to any preceding paragraph, wherein the anti-IL-13 R antibody comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
56. The antibody or antigen binding fragment thereof, method or use according to preceding paragraph, wherein the anti-IL-13 R antibody comprises a VL domain comprising an amino acid sequence shown in SEQ ID NO: 53 or a sequence at least 95% identical thereto.
57. The antibody or antigen binding fragment thereof, method or use according to any preceding paragraph, wherein the antibody is provided as a pharmaceutical formulation comprising: 10 to 140 mg/ml of the antibody or binding fragment; 50 mM to 150 mM of arginine (for example 50, 55, 60, 65, 70, 75, 80, 85, 90, 95, 100, 105, 110, 115, 120, 125, 130, 135, 140, 145 or 150, such as 100 mM arginine); 15 to 25 mM histidine buffer, for example 15, 16, 17, 18, 19, 20, 21, 22, 23, 24 and 25, such as 20 mM histidine buffer; 0.01-0.03% of a non-ionic surfactant, such as 0.02% w/v and wherein the pH of the formulation is in the range 5.5 to 7.5 for example 6.2 to 7.2 (such as 6.2, 6.3, 6.4, 6.5, 6.6, 6.7, 6.8, 6.9, 7.0, 7.1, 7.2), such as 6.5 to 7.0, in particular 6.4 to 6.9)
58. The antibody or antigen binding fragment thereof, method or use according to paragraphs 57, wherein the osmolarity of the formulation is in the range 350 to 550 mOsmo/kg, for example 350, 355, 365, 370, 375, 380, 385, 390, 395, 400, 405, 410, 415, 420, 425, 430, 435, 440, 445, 450, 455, 460,,465, 470, 475, 480, 485, 490, 495, 500, 505, 515, 520, 525, 530, 535, 540, 545, 550, such as 405 to 435 mOsmo/kg.
59. The antibody or antigen binding fragment thereof, method or use according to paragraph 57or 58, which further comprises 50 to 200 mM of a sugar, for example 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, such as 180 mM sugar.
60. The antibody or antigen binding fragment for use according to any one of paragraphs 57 to 59, wherein the pH is 6.5.
61. The antibody or antigen binding fragment thereof for use according to any one of paragraphs 57 to 60, wherein the formulation does not comprise NaCl.
62. The antibody or antigen binding fragment thereof for use according to any one of paragraphs 57 to 61, wherein the formulation comprises 50 to 150 mM of NaCl, for example50, 55, 60, 65, 70, 75, 80, 85, 90, 95, 100, 105, 110, 115, 120, 125, 130, 135, 140, 145, 150, such as 62.5or 140 mM NaCl.
63. Use of an antibody or antigen binding fragment, which is an inhibitor of signalling through of the IL-13 Rα1 by binding the said receptor, according to any one of paragraphs 1 to 58, for the manufacture of a medicament for the treatment of atopic dermatitis wherein the incidence of side effects in the eyes are reduced in comparison to treatment with the therapeutic dose of dupilumab for treatment of the same.

In one embodiment, the moderate to severe atopic dermatitis (such as severe AD), is lesional atopic dermatitis.

Also provided is method of treating a patient for atopic dermatitis (for example moderate to severe atopic dermatitis, in particular poorly controlled moderate to severe atopic dermatitis) according to the present disclosure comprising administering an antibody or antigen binding fragment thereof, or pharmaceutical formulation disclosed herein.

Poorly controlled as employed herein refers to poor controlled by existing approved medicaments, for example topical medicine, oral medicines and/or biological medicines such as dupilumab (in particular topical medicine or dupilumab).

The results further suggest that Eblasakimab has a comparable or in some cases a higher efficacy compared to Dupilumab, thus demonstrating the potential of ablasakimab as an alternative therapy for the treatment and management of atopic dermatitis, in particular for patients that have previously taken dupilumab.

Thus, in one embodiment there is provide treatment of a patient population where the patient had poorly controlled symptoms and/or adverse side effects when treated with dupilumab i.e. the patient population is those who received prior treatment with dupilumab, in particular a patient that has failed with dupilumab.

In further aspect there is provided use of an antibody or antigen binding fragment or an pharmaceutical formulation disclosed herein for use in the manufacture of a medicament for the treatment of atopic dermatitis according to the present disclosure.

In one embodiment there is a provided a reduction in the Investigator Global Assessment IGA with/after treatment according to the present disclosure, for example an assessment of 0, 1 or 2, (no inflammatory signs, almost clear and mild disease respectively). In particular there is provided an IGA score of 0 or 1.

In one embodiment combination therapy is employed comprising the antibody, antigen binding fragment thereof or a formulation according to the present disclosure and a further medicament.

In one embodiment the further medicament is for the treatment of atopic dermatitis, for example topical steroids, oral steroids, and/or antihistamines.

Surprisingly disease modification following treatment with an anti IL-13Rα1 antibody or binding fragment thereof according to the present disclosure closely follows reduction in TARC, in fact the TARC reduction and EASI reduction correlate closely in treatment according to the present disclosure.

Disease modification as employed herein relates to improvements in the disease status, in particular a reduction in the EASI score. In one embodiment this reduction is maintained for at least a period even after treatment is stopped.

Baseline as employed herein refers to the levels before treatment according to the present disclosure commences. Generally, the baseline is measured and/or recorded.

Loading dose as employed herein refers to a dose higher than the “normal doses” in the treatment cycle. A loading dose is usually employed to load the patient's system with drug so that when subsequent doses are administered the levels in the requisite tissue are maintained at or above a minimum predefined level.

“About day” as employed herein means administration approximately at the day, for example +/−1, 2, 3, 4, 5, 6 or 7 days. Doses early in the treatment cycle may need to be administered close to the specified day, for example +/−1 day. Later doses may be allowed more latitude i.e. +/−up to 7 days.

Atopic dermatitis as employed herein refers to inflammation of the skin and includes: dry/itchy skin and red rashes.

Moderate to severe atopic dermatitis are defined by one or more, such as all the parameters defined herein.

Severe atopic dermatitis includes very severe atopic dermatitis. Very severe atopic dermatitis is a subset of atopic dermatitis.

Investigator's global assessment (IGA) as used herein refers to a tool for the assessment of atopic dermatitis. It uses a 0-5 point scale depending on the severity of a patient's symptoms:

| Score | Severity | Description of symptoms |
|---|---|---|
| 0 | clear | no inflammatory signs |
| 1 | almost clear | Just perceptible erythema and just perceptible |
| | | papulation/infiltration |
| 2 | mild disease | Mild erythema and mild papulation/infiltration |
| 3 | moderate disease | moderate erythema and moderate papulation/infiltration |
| 4 | severe disease | severe erythema and severe papulation/infiltration |
| 5 | very severe disease | severe erythema and severe papulation/infiltration with |
| | | oozing/crusting |

Interleukin-13 receptor (IL-13 R) as used herein is a type I cytokine receptor, which binds to Interleukin-13. It consists of two subunits, encoded by IL13Rα1 and IL4R, respectively. These two genes encode the proteins IL-13 Rα1 and IL-4Rα. These form a dimer with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STAT6. Human IL-13 Rα1 has the Uniprot number P3597.

IL-13 Rα2, previously called IL-13 R and IL-13 Rα, is another receptor which is able to bind to IL-13. However, in contrast to IL-13 Rα1, this protein binds IL-13 with high affinity, but it does not bind IL-4. Human IL-13 Rα2 has the Uniprot number Q14627.

In one embodiment CDRH1 comprises an amino acid sequence GYSFTSYWIG (SEQ ID NO: 1). In one embodiment CDRH2 comprises an amino acid sequence VIYPGDSYTR (SEQ ID NO: 2).

In one embodiment CDRH3 comprises the formula:

SEQ ID NO: 3
X₁ Pro Asn Trp Gly X₆ X₇ Asp X₉

X₁ denotes Phe, Met, Gln, Leu or Val
X₆ denotes Ser or Ala
X₇ denotes Phe, Leu, Ala or Met
X₉ denotes Tyr, Gln, Lys, Arg, Trp, His, Ala, Thr, Ser, Asn or Gly

In one embodiment the IL13-R1α1 antibody or binding fragment employed in the formulation of the present disclosure comprises a CDRH3 independently selected from a sequence comprising SEO ID NO. 4 to 30.

| SEQ ID NO: | CDR H3 SEQUENCE |
|---|---|
| 4 | FPNWGALDQ |
| 5 | VPNMGSLDT |
| 6 | FPNWGSMDA |
| 7 | FPNWGSLDH |
| 8 | MPNWGSFDY |
| 9 | MPNWGSFDT |
| 10 | MPNWGSLDH |
| 11 | MPNWGSFDS |
| 12 | MPNWGSLDT |
| 13 | MPNWGSLDA |
| 14 | MPNWGSLDN |
| 15 | MPNWGALDS |
| 15 | MPNWGSLDN |
| 17 | MPNWGSLDY |
| 18 | MPNWGSFDH |
| 19 | MPNWGSLDS |
| 20 | MPNWGSLDG |
| 21 | VPNWGSLDG |
| 22 | CARFPNWGSLDHWGQGTLVTVSSASIKG |
| 23 | CARMPNWGSLDHWGQGTLVTVSSASTKG |
| 24 | CARMPNWGSFDYWGQGTLVTVSSASIKG |
| 25 | VRMPNWGSLDHW |
| 26 | VRMPNWGSLDHWGQGTLVTVSSADIKG |
| 27 | ARMPNWGSLDHWGQGTLVTVSSASIKG |
| 28 | FPNWGSFDYWGQGTLVTVSSASIKG |
| 29 | VPNWGSLDA |
| 30 | FPNWGSFDY |

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3comprising an amino acid sequence as set forth in SEQ ID NO: or 3.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.In one embodiment CDRL1 is an amino acid sequence comprising RASQSISSSYLA SEQ ID NO: 31.

In one embodiment CDRL2 is an amino acid sequence comprising GASSRAT SEQ ID NO: 32In one embodiment CDL3 comprises the formula:

SEQ ID NO: 33
Gln X₂X₃X₄X₅

X₂ denotes Gln, Arg, Met, Ser, Thr or Val.
X₃ denotes Tyr or Val.
X₄ denotes Glu, Ala, Gly or Ser.
X₅ denotes Thr, Ala or Ser. In one embodiment the IL-13 Rα1 antibody employed in the formulation of the present disclosure comprises a CDRL3 independently selected from a sequence comprising SEQ ID NO: 34 to 47:

| SEQ ID NO: | CDR L3 SEQUENCE |
|---|---|
| 34 | QRYAT |
| 35 | QRYST |
| 36 | QMYST |
| 37 | QQVGT |
| 38 | QQVST |
| 39 | QQYST |
| 40 | QSYST |
| 41 | QQYAT |
| 42 | QQYSS |
| 43 | QTYST |
| 44 | QQYGS |
| 45 | QQYAS |
| 46 | QQYEA |
| 47 | QQYET |

In one embodiment, the anti-IL-13 Rα antibody or binding fragment employed in the present disclosure comprises a CDRL1 comprising an amino acid sequence SEQ ID NO: 31, a CDRL2comprising an amino acid sequence SEQ ID NO: 32, and a CDRL3 comprising an amino acid sequence as set forth in SEQ ID NO: 33.

In one embodiment, the anti-IL-13 Rα antibody of the present disclosure comprises a VL CDR1 comprising an amino acid sequence SEQ ID NO: 84, a VL CDR2 comprising an amino acid sequence SEQ ID NO: 85, and a VL CDR3 comprising an amino acid sequence as set forth in SEQ ID NO: 34 35, 36, 37, 38, 39, 40, 41, 42, 43, 44, 45, 46, or 47.

In one embodiment, the anti-IL-13 Rα antibody of the present disclosure comprises a CDRL1comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: or 3, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 33.

In one embodiment, the anti-IL13R antibody of the present disclosure comprises a CDRH1comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2comprising an amino acid sequence SEQ ID NO: 32, and a CDRL3 comprising an amino acid sequence as set forth in SEQ ID NO: 34, 35, 36, 37, 38, 39, 40, 41, 42, 43, 44, 45, 46, or 47.

In one embodiment, the anti-IL13R antibody of the present disclosure comprises a CDRH1comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 3 or 10, a CDRL1 comprising an amino acid sequence SEQ ID NO: 31, a CDRL2comprising an amino acid sequence SEQ ID NO: 32, and a CDRL3 comprising an amino acid sequence as set forth in SEQ ID NO: 45.

In one embodiment, the anti-IL13R antibody of the present disclosure comprises a CDRH1comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment the VH region is independently selected from a sequence comprising:

SEQ ID NO: 48
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
FPNWGSFDYWGQGTLVTVSS
SEQ ID NO: 49
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSFDYWGQGTLVTVSS
SEQ ID NO: 50
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCVR
MPNWGSLDHWGQGTLVTVSS
SEQ ID NO: 51
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSS

or a sequence at least 95% identical to any one of the same. In one embodiment the VL is independently selected from a sequence comprising:

SEQ ID NO: 52
EIVLTQSPGTLSLSPGERATLSCRASQSISSSYLAWYQQKPGQAPRLLIY
GASSRATGIPDRFSGSGSGTDFTLTISRLEPEDFAVYYCQQYETFGQGTK
VEI*
SEQ ID NO: 53
EIVLTQSPGTLSLSPGERATLSCRASQSISSSYLAWYQQKPGQAPRLLIY
GASSRATGIPDRFSGSGSGTDFTLTISRLEPEDFAVYYCQQYASFGQGTK
VEI*
SEQ ID NO: 54
EIVLTQSPGTLSLSPGERATLSCRASQSISSSYLAWYQQKPGQAPRLLIY
GASSRATGIPDRFSGSGSGTDFTLTISRLEPEDFAVYYCQQYEAFGQGTK
VEI*
(*K deleted in a post translational modification).

or a sequence at least 95% identical to any one of the same (* K deleted in a post translational modification).

In one embodiment the VH sequence is SEQ ID NO: 48 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VH sequence is SEQ ID NO: 49 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VH sequence is SEQ ID NO: 50 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VH sequence is SEQ ID NO: 51 (or a sequence at least 95% identical thereto) and the VL sequence is SEQ ID NO: 52, SEQ ID NO: 53, SEQ ID NO: 54 or SEQ ID NO: 55 (or a sequence at least 95% identical to any one of the same).

In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51. (or a sequence at least 95% identical to any one of the same)

In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VL sequence is SEQ ID NO: 55 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VH sequence is SEQ ID NO: 51 (or a sequence at least 95% identical thereto) and the VL sequence is SEQ ID NO: 53 ((or a sequence at least 95% identical thereto). Variable region as employed herein refers to the region in an antibody chain comprising the CDRs and a suitable framework.

In one embodiment the heavy chain comprises a sequence independently selected from:

SEQ ID NO: 56
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSFDYWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLG*
SEQ ID NO: 57
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCVR
MPNWGSLDHWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLG*
SEQ ID NO: 58
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCVR
MPNWGSLDHWGQGTLVTVSSASIKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLG*
SEQ ID NO: 59
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLG*
SEQ ID NO: 60
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSSASIKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLG*
SEQ ID NO: 61
EVQLVQSGAEVKKPGESLKISCKGSGYSFTSYWIGWVRQMPGKGLEWMG
VIYPGDSYTRYSPSFQGQVTISADKSISTAYLQWSSLKASDTAMYYCAR
MPNWGSLDHWGQGTLVTVSSASTKGPSVFPLAPCSRSTSESTAALGCLV
KDYFPEPVTVSWNSGALTSGVHTFPAVLQSSGLYSLSSVVTVTSSNFGT
QTYTCNVDHKPSNTKVDKTVERKCCVECPPCPAPPVAGPSVFLFPPKPK
DTLMISRTPEVTCVVVDVSQEDPEVQFNWYVDGVEVHNAKTKPREEQFN
STFRVVSVLTVLHQDWLNGKEYKCKVSNKGLPSSIEKTISKTKGQPREP
QVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PG*,

or a sequence at least 95% identical to any one of the same (*K deleted in a post translational modification)

In one embodiment the light chain is independently selected from:

SEQ ID NO: 62
EIVLTQSPGTLSLSPGERATLSCRASQSISSSYLAWYQQKPGQAPRLLI
YGASSRATGIPDRFSGSGSGTDFTLTISRLEPEDFAVYYCQQYASFGQG
TKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC
SEQ ID NO: 63
EIVLTQSPGTLSLSPGERATLSCRASQSISSSYLAWYQQKPGQAPRLLI
YGASSRATGIPDRFSGSGSGTDFTLTISRLEPEDFAVYYCQQYEAFGQG
TKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC
SEQ ID NO: 64
EIVLTQSPGTLSLSPGERATLSCRASQSISSSYLAWYQQKPGQAPRLLI
YGASSRATGIPDRFSGSGSGTDFTLTISRLEPEDFAVYYCQQYETFGQG
TKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQ
GLSSPVTKSFNRGEC

or a sequence at least 95% identical to any one of the same.

In one embodiment the heavy chain is independently selected from SEQ ID NO: 56, 57, 58,59, 60 and 61 (or a sequence at least 95% identical to any one of the same) and the light chain is independently selected from SEQ ID NO: 62, 63 and 64 (or a sequence at least 95% identical to any one of the same).

In one embodiment the heavy chain is SEQ ID NO: 56 (or a sequence at least 95% identical thereto) and the light chain is independently selected from SEQ ID NO: 62, 63 and 64 (or a sequence at least 95% identical to any one of the same).

In one embodiment the heavy chain is SEQ ID NO: 57 (or a sequence at least 95% identical thereto) and the light chain is independently selected from SEQ ID NO: 62, 63 and 64 (or a sequence at least 95% identical to any one of the same).

In one embodiment the heavy chain is SEQ ID NO: 58 (or a sequence at least 95% identical thereto) and the light chain is independently selected from SEQ ID NO: 62, 63 and 64 (or a sequence at least 95% identical to any one of the same).

In one embodiment the heavy chain is SEQ ID NO: 59 (or a sequence at least 95% identical thereto) and the light chain is independently selected from SEQ ID NO: 62, 63 and 64 (or a sequence at least 95% identical to any one of the same).

In one embodiment the heavy chain is SEQ ID NO: 60 (or a sequence at least 95% identical thereto) and the light chain is independently selected from SEQ ID NO: 62, 63 and 64 (or a sequence at least 95% identical to any one of the same).

In one embodiment the heavy chain is SEQ ID NO: 61 (or a sequence at least 95% identical thereto) and the light chain is independently selected from SEQ ID NO: 62, 63 and 64 (or a sequence at least 95% identical to any one of the same).

In one embodiment the heavy chain is SEQ ID NO: 59 or 61 (or a sequence at least 95% identical to any one of the same) and a light chain with the sequence shown in SEQ ID NO: 62 (or a sequence at least 95% identical thereto).

In one embodiment the heavy chain is SEQ ID NO: 59 (or a sequence at least 95% identical to any one of the same) and a light chain with the sequence shown in SEQ ID NO: 62 (or a sequence at least 95% identical thereto).

In one embodiment the heavy chain is SEQ ID NO: 61 (or a sequence at least 95% identical to any one of the same) and a light chain with the sequence shown in SEQ ID NO: 62 (or a sequence at least 95% identical thereto).

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Any discussion of a protein, antibody or amino acid sequence herein will be understood to include any variants of the protein, antibody or amino acid sequence produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” (C-terminal lysine residues of encoded antibodies are often removed during the manufacturing process) and/or have part or all of a signal sequence incompletely processed and, as a consequence, remain at the terminus of the antibody. It is understood that an antibody comprising a particular amino acid sequence or binding fragment thereof may be a heterogeneous mixture of the stated or encoded sequence and/or variants of that stated or encoded sequence or binding fragment thereof.

In one embodiment the present disclosure extends to a sequence explicitly disclosed herein where the C-terminal lysine has been cleaved.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089;WO91/09967). It will be appreciated that it may only be necessary to transfer the specificity determining residues of the CDRs rather than the entire CDR (see for example, Kashmiri et al., 2005,Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al.,). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO91/09967.

In one embodiment the anti-IL13R antibodies of the present disclosure are fully human, in particular one or more of the variable domains are fully human.

Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073 B1, U.S. Pat. Nos. 5,545,806, 5,569,825, 5,625,126, 5,633,425, 5,661,016, 5,770,429, EP 0438474 and EP0463151.

Constant region as employed herein is intended to refer to the constant region portion located between two variable domains, for example non-cognate variable domains, in the heavy chain. Thus, the presently disclosed anti-IL13R antibody may comprise one or more constant regions, such as a naturally occurring constant domain or a derivate of a naturally occurring domain. A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or via a coupling agent to the effector molecule. Techniques for conjugating such effector molecules to antibodies are well known in the art (see, Hellstrom et al., Controlled Drug Delivery, 2nd Ed., Robinson et al., eds., 1987, pp. 623-53;Thorpe et al., 1982, Immunol. Rev., 62:119-58 and Dubowchik et al., 1999, Pharmacology and Therapeutics, 83, 67-123). Particular chemical procedures include, for example, those described in WO 93/06231, WO 92/22583, WO 89/00195, WO 89/01476 and WO03031581. Alternatively, where the effector molecule is a protein or polypeptide the linkage may be achieved using recombinant DNA procedures, for example as described in WO 86/01533 and EP0392745.

The term effector molecule as used herein includes, for example, biologically active proteins, for example enzymes, other antibody or antibody fragments, synthetic or naturally occurring polymers, nucleic acids and fragments thereof e.g. DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 1251, 1311, 111 In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo-or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly (ethyleneglycol), poly (propyleneglycol) poly (vinylalcohol) or derivatives thereof, especially optionally substituted poly (ethyleneglycol) such as methoxypoly (ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly (ethyleneglycol) or, especially, a methoxypoly (ethyleneglycol) or a derivative thereof, and especially with a molecular weight in the range from about 15000 Da to about 40000 Da.

In one example antibodies for use in the present invention are attached to poly (ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In one embodiment the formulation herein is administered in combination with another therapy.

“In combination” as employed herein is intended to encompass where the anti-IL13R antibody is administered before, concurrently with another therapy.

Therapy as employed herein refers to amelioration of symptoms or conditions of a disease including stabilising disease and/or sending disease into remission, rendering flares less likely to occur and similar, in particular without eliciting dose limiting side effects. Suitable therapeutic doses are generally a balance between therapeutic effect and tolerable toxicity, for example where the side-effect and toxicity are tolerable given the benefit achieved by the therapy. In an independent aspect there is provided an antibody or antigen binding fragment thereof, which is an inhibitor of signalling through of the IL-13Rα1 by binding the said receptor, for use in the treatment of atopic dermatitis (moderate, severe or very severe atopic dermatitis in particular poorly controlled moderate to severe atopic dermatitis) by parenteral administration of a treatment cycle comprising a dose in the range 200 mg to 600 mg, (such as 400 to 600 mg), wherein occurrence conjunctivitis is minimised, for example as detailed herein.

Conjunctivitis can be a side effect of biological treatments for atopic dermatitis. Advantageously, such side effects are minimised in therapy according to the present disclosure, in particular no such side effects were observed with Eblasakimab treatment.

In one embodiment a formulation according to the present disclosure (including a formulation comprising same) is administered monthly, for example in a treatment cycle or as maintenance therapy.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section of this specification contains relevant technical information and may be used as basis for amendment.

Subject headings herein are employed to divide the document into sections and are not intended to be used to construe the meaning of the disclosure provided herein.

The present invention is further described by way of illustration only in the following examples.

### SUMMARY OF THE FIGURES

FIG. 1A shows patient demographics of full analysis set

FIG. 1B shows base line disease characteristics of the full analysis set

FIG. 1C shows base line characteristics of data evaluable for efficacy

FIG. 2A shows % change from baseline in EASI score at day 57

FIG. 2B shows % change from baseline in EASI score at day 57.

FIG. 2C shows % change from baseline in EASI score at day 57

FIG. 3A shows % change from baseline in EASI score at day 29

FIG. 3B shows % change from baseline in EASI score at day 29

FIG. 4A shows % change from baseline in EASI score over time

FIG. 4B shows % change from baseline in EASI score over time

FIG. 5A shows % change from baseline in EASI score over time for individual patients

FIG. 5B shows % change from baseline in EASI score over time for individual patients 200 mg

FIG. 5C show % change from baseline in EASI score over time for individual patients 400 mg

FIG. 5D show % change from baseline in EASI score over time for individual patients 600 mg

FIG. 6A shows day 57 sensitivity analysis in the mITT

FIG. 6B shows sensitivity analysis in the mITT (200, 400 and 600 mg)

FIG. 6C shows analysis it in the mITT (low dose and high dose)

FIG. 7A shows EASI 50, EASI 75 and EASI 90 at day 57

FIG. 7B shows EASI 50 at day 57 (200, 400 and 600 mg)

FIG. 7C shows EASI 50 at day 57 (low and high dose)

FIG. 7D shows EASI 75 at day 57 (200, 400 and 600 mg)

FIG. 7E shows EASI 75 at day 57 (low and high dose)

FIG. 7F shows EASI 90 at day 57 (200, 400 and 600 mg)

FIG. 7G shows EASI 90 at day 57 (low and high dose)

FIG. 8A show % EASI 50 reduction over time

FIG. 8B shows % EASI 75 reduction over time

FIG. 8C shows % EASI 90 reduction over time

FIG. 9 shows sensitivity analysis in the Mitt

FIG. 10A shows the proportion of patients with IGA score 0 or 1 at day 57 in a summary table

FIG. 10B shows the proportion of patients with IGA score 0 or 1 at day 57

FIG. 10C shows the proportion of patients with IGA score of 0 or 1

FIG. 11 shows baseline TARC and IgE of patients

FIG. 12A show average % change from baseline TARC (200 mg and 400 mg)

FIG. 12B show average % change from baseline TARC (400 mg and placebo)

FIG. 12C show % change from baseline TARC for individual patients

FIG. 13A shows IgE % change from baseline (200 mg and 400 mg)

FIG. 13B shows average IgE % change from baseline (200 mg and 400 mg)

FIG. 13C shows % IgE change from baseline for 3 individual patients

FIG. 13D shows % IgE change from baseline for 4 individual patients on 200 mg

FIG. 13E shows % IgE change from baseline for 6 individual patients on 400 mg

FIG. 14 shows eblasakimab exposure, average EASI score, average TARC levels and average Ige levels.

FIG. 15 shows eblasakimab efficacy versus dupilumab efficacy

FIG. 16 shows median percentage change from baseline in IgE dupilumab

FIG. 17 shows % change from baseline for dupiluman 300 mg weekly dosing

FIG. 18 shows staining intensity for IL-13 Rα1 expression in healthy skin, non-lesional atopic dermatitis and lesional atopic dermatitis

FIG. 19 shows staining IL-13 Rα1 expression on mast cells and eosinophils for ealthy skin, non-lesional atopic dermatitis and lesional atopic dermatitis

FIG. 20 shows gene expression for type I and type II IL-13 receptors

### EXAMPLES

### Example 1

### Study Protocol (Initial MAD Escalation)

Patients enrolled in ascending dose cohorts of EBLASAKIMAB (SEQ ID NO: 51, 53 and 59 herein): 200 mg, 400 mg, 600 mg. ASLAN low dose=EBLASAKIMAB 200 mg, ASLAN high dose=EBLASAKIMAB 400 mg+EBLASAKIMAB 600 mg.

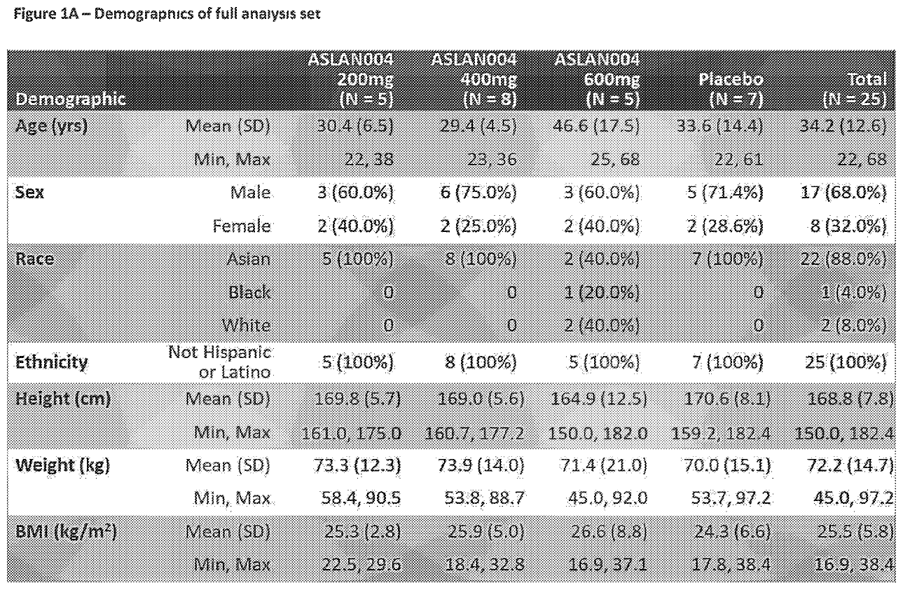

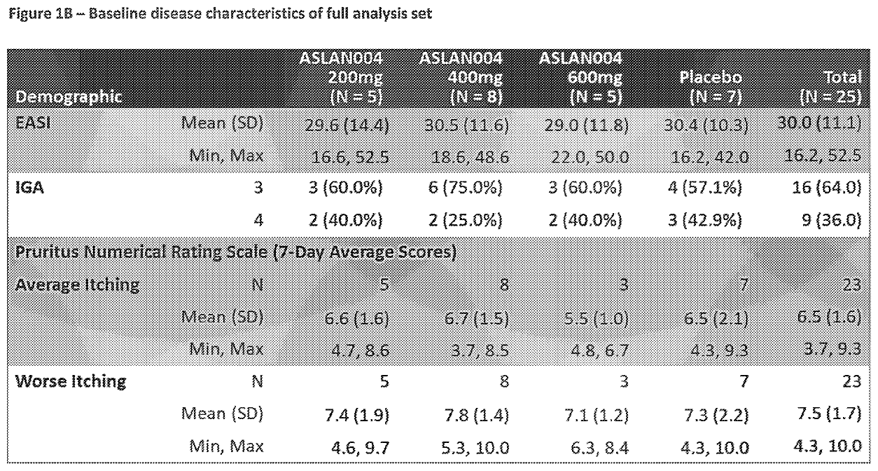

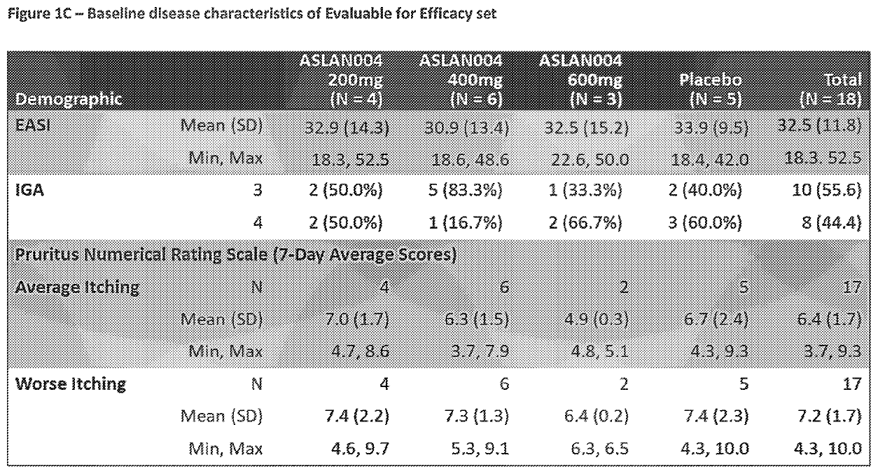

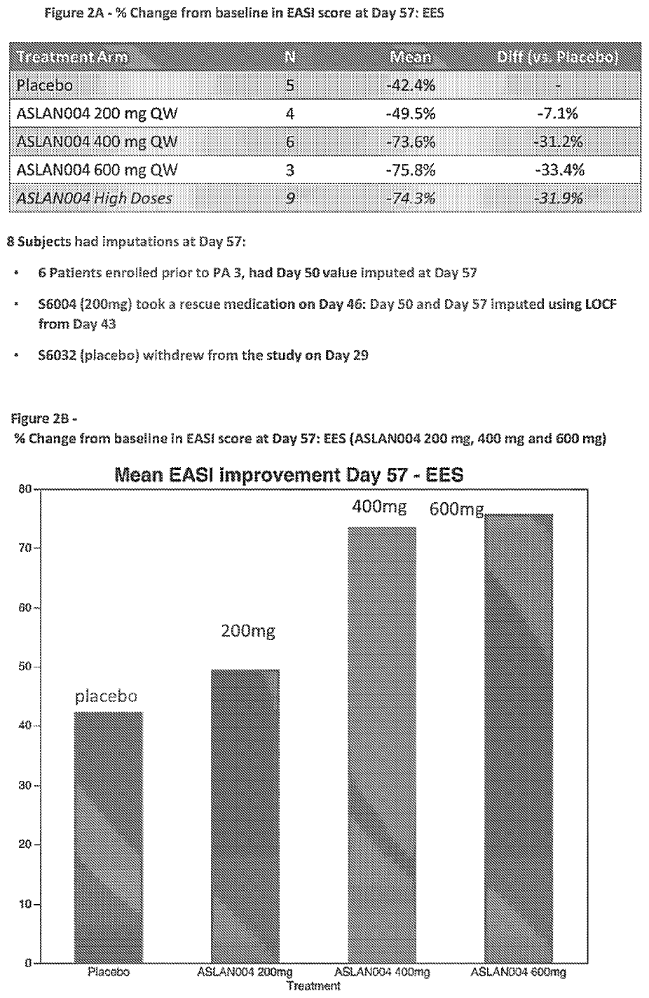

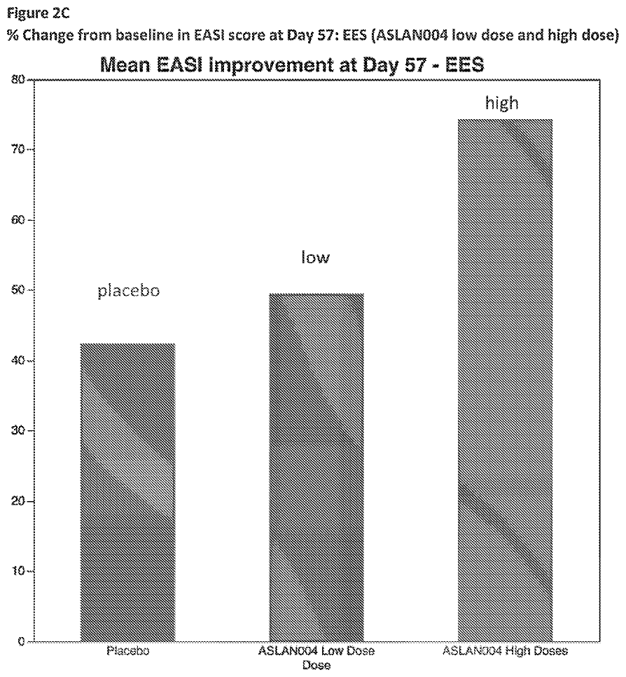

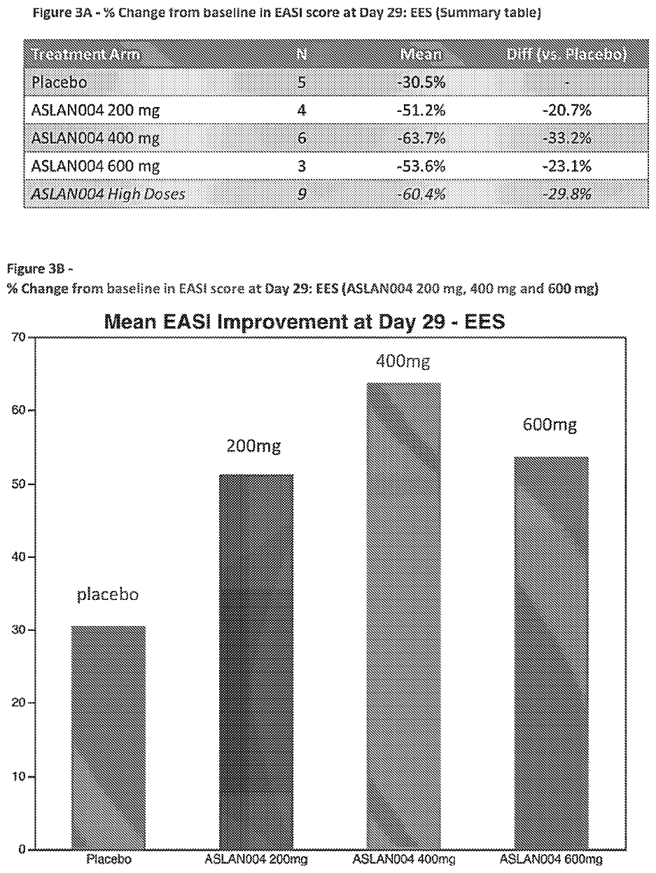

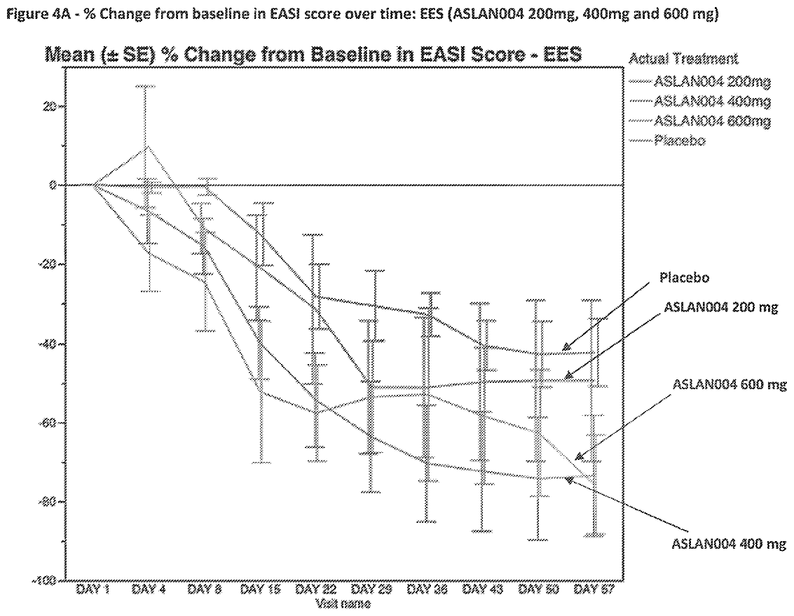

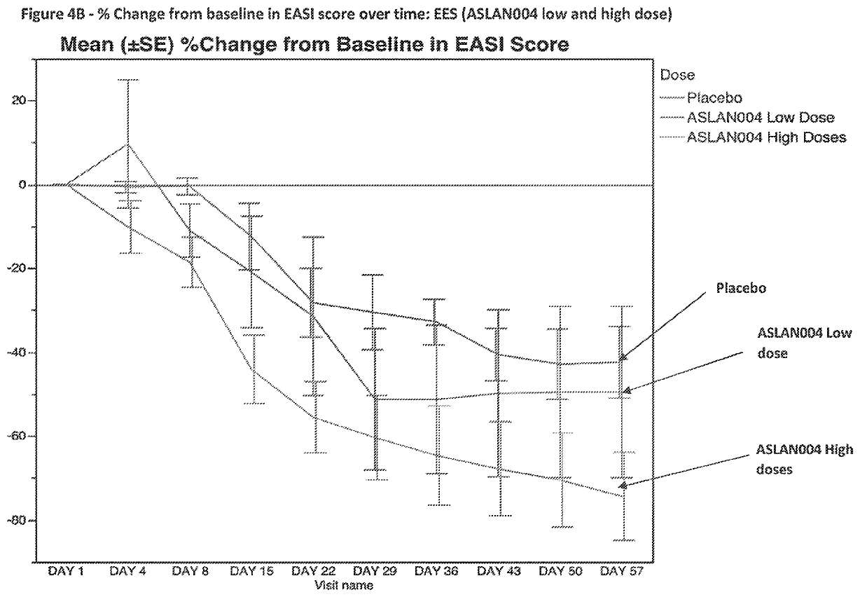

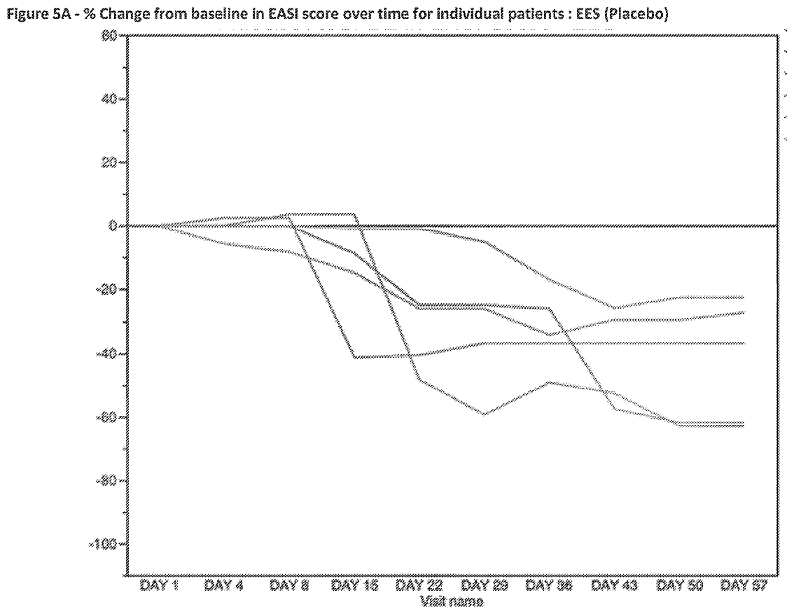

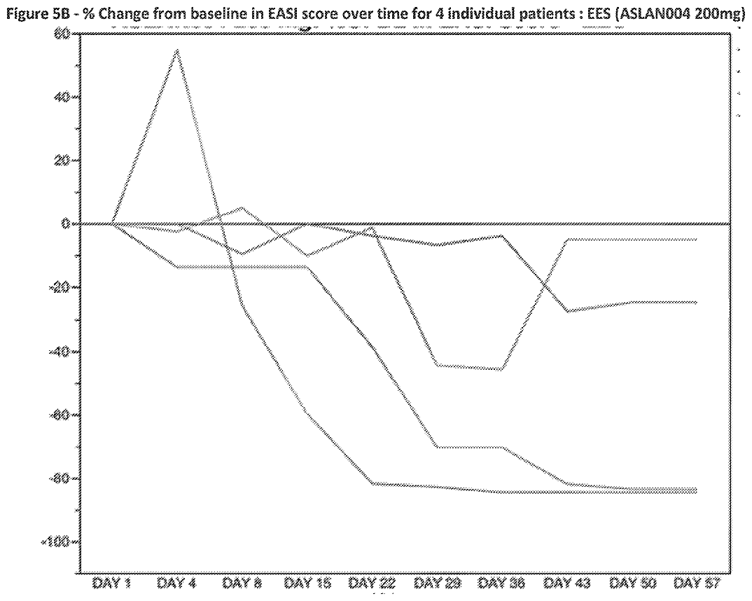

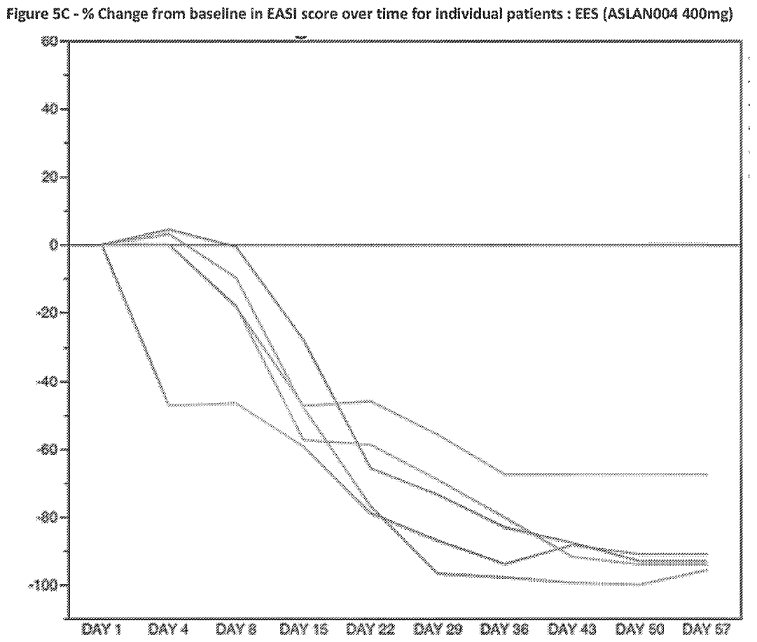

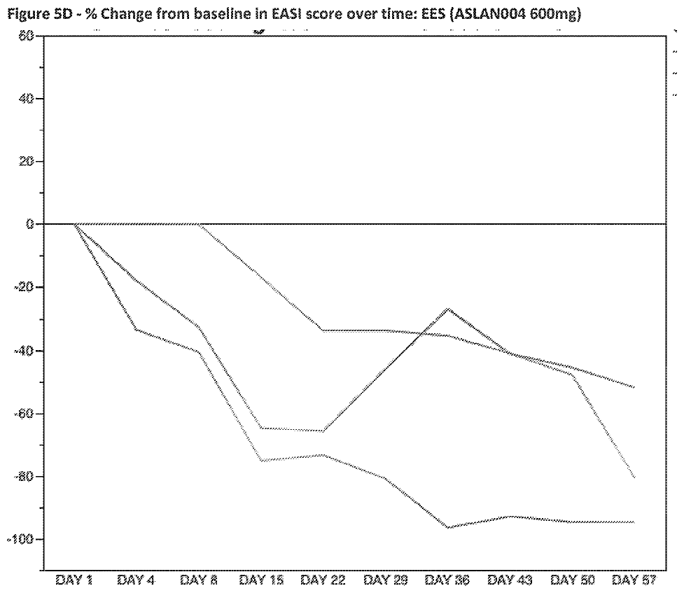

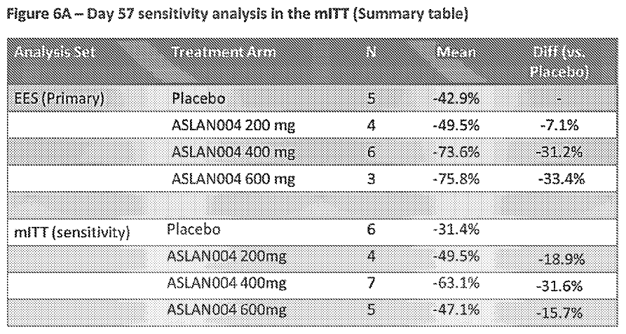

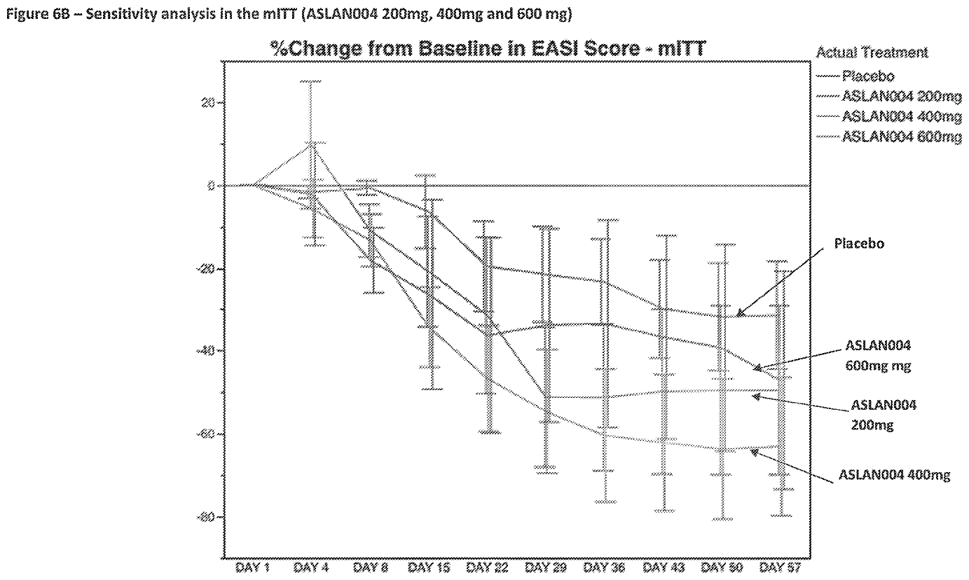

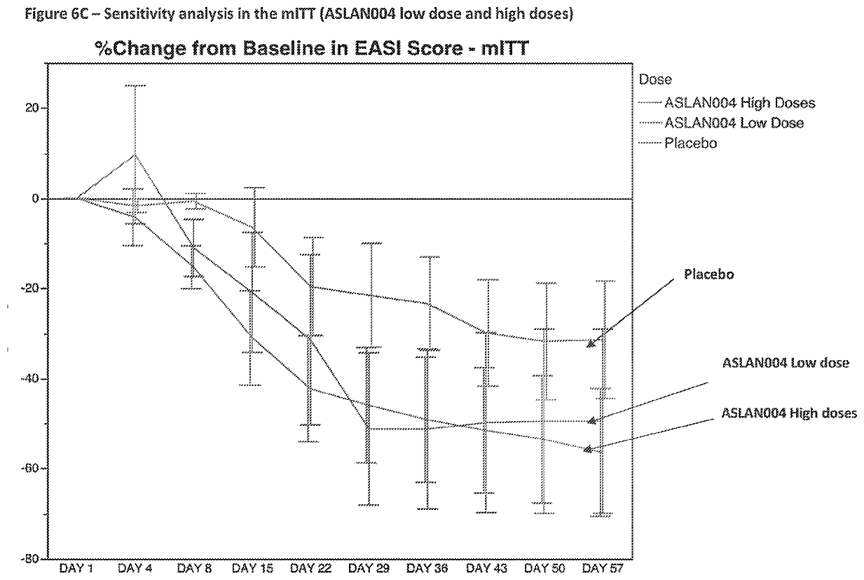

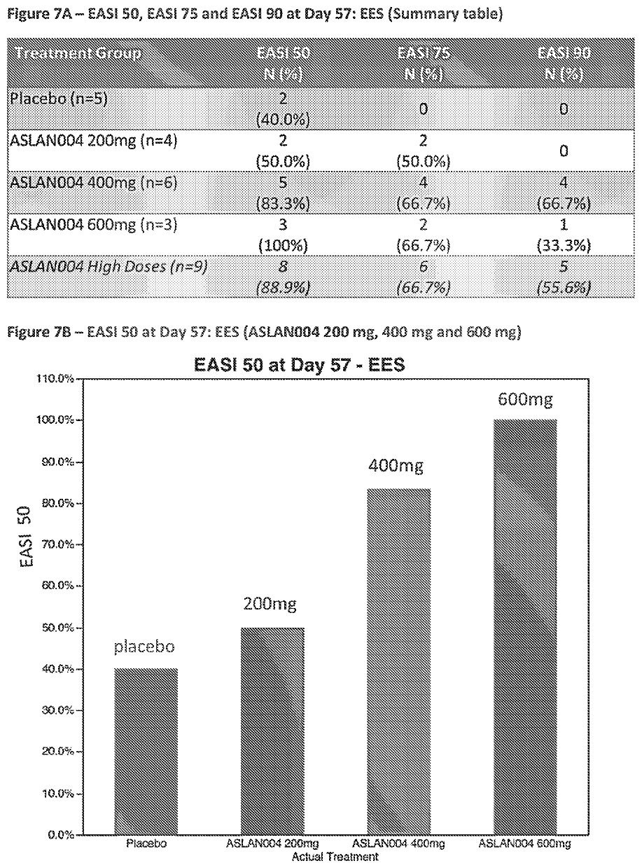

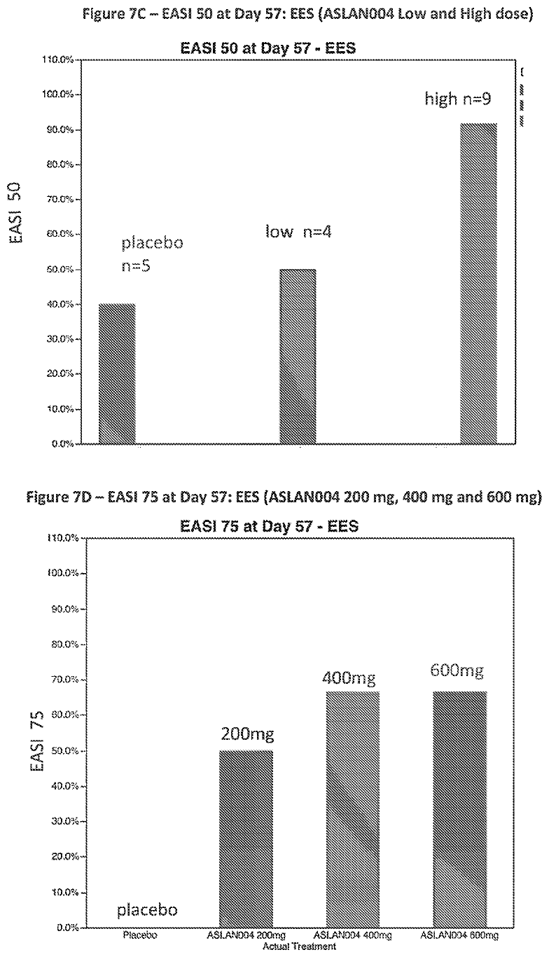

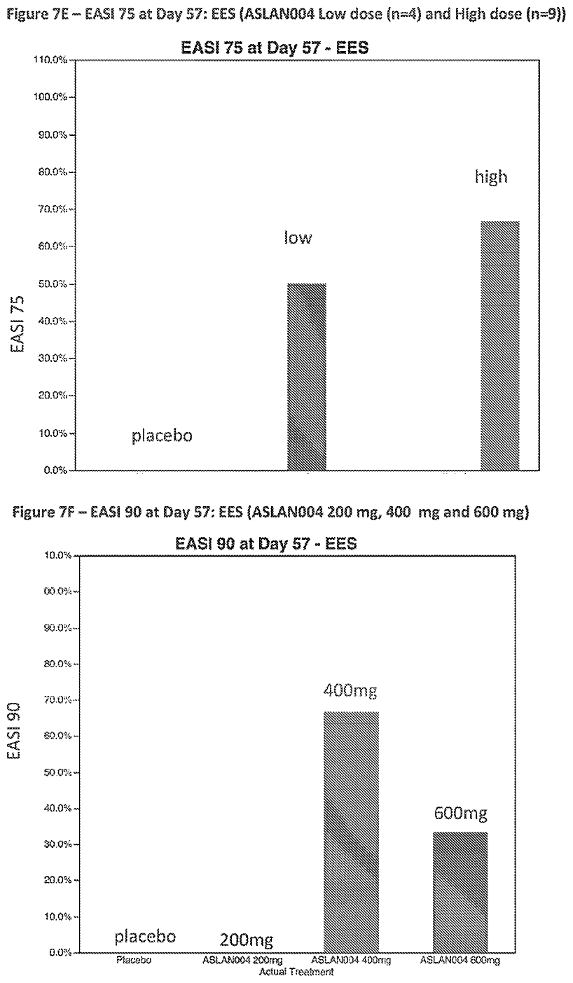

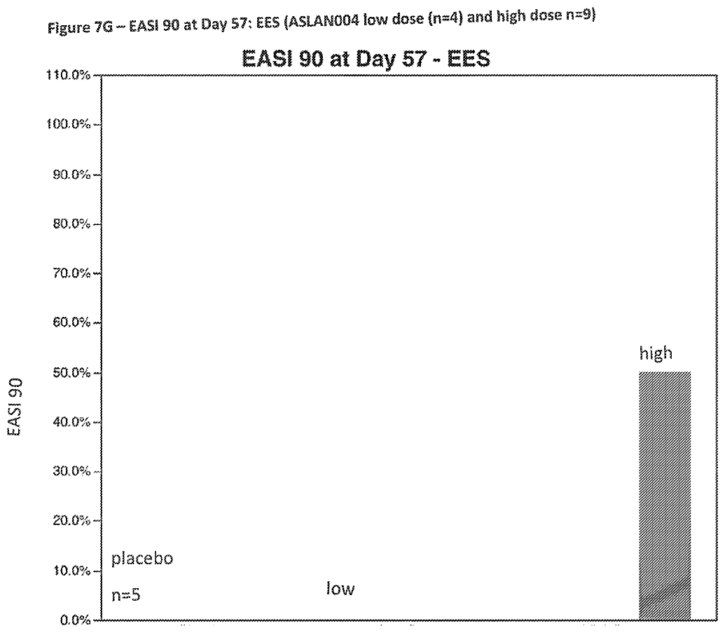

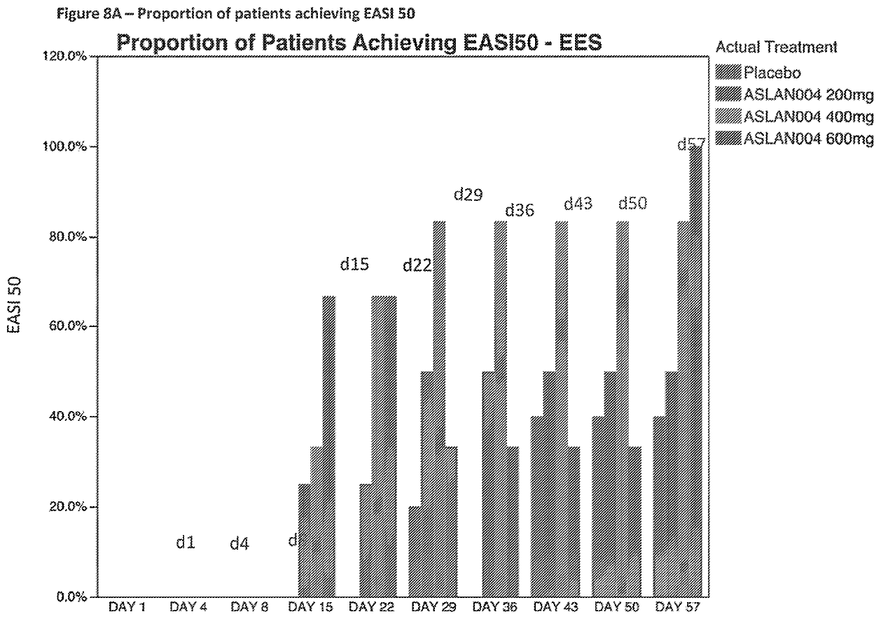

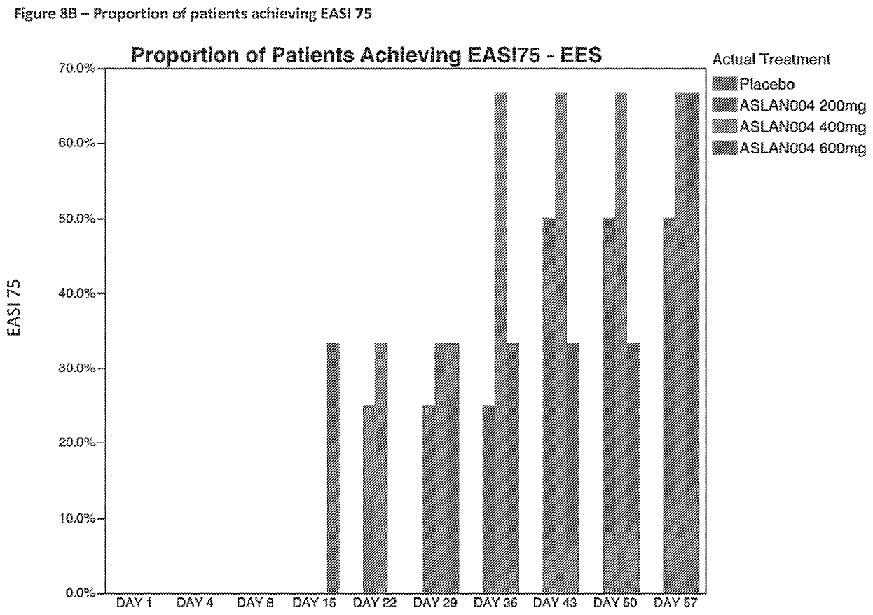

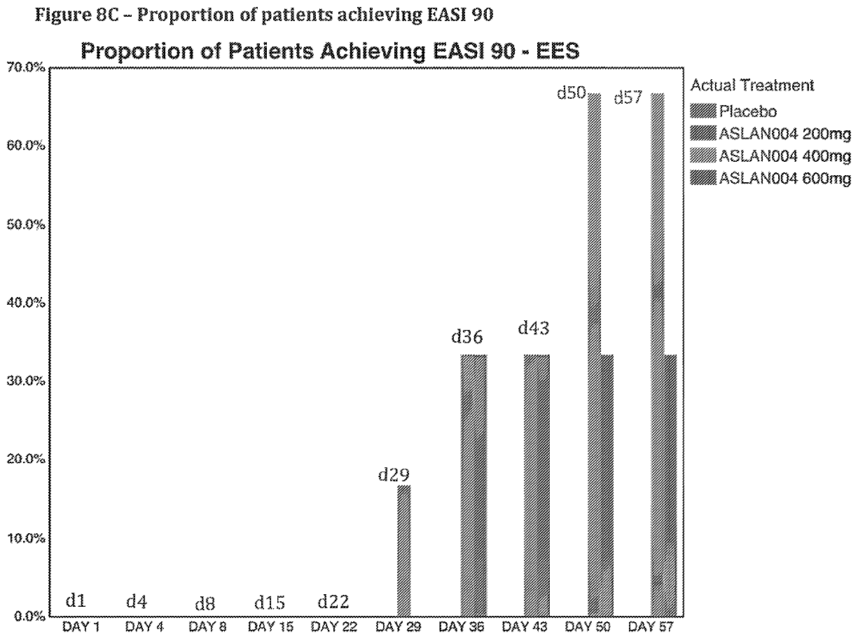

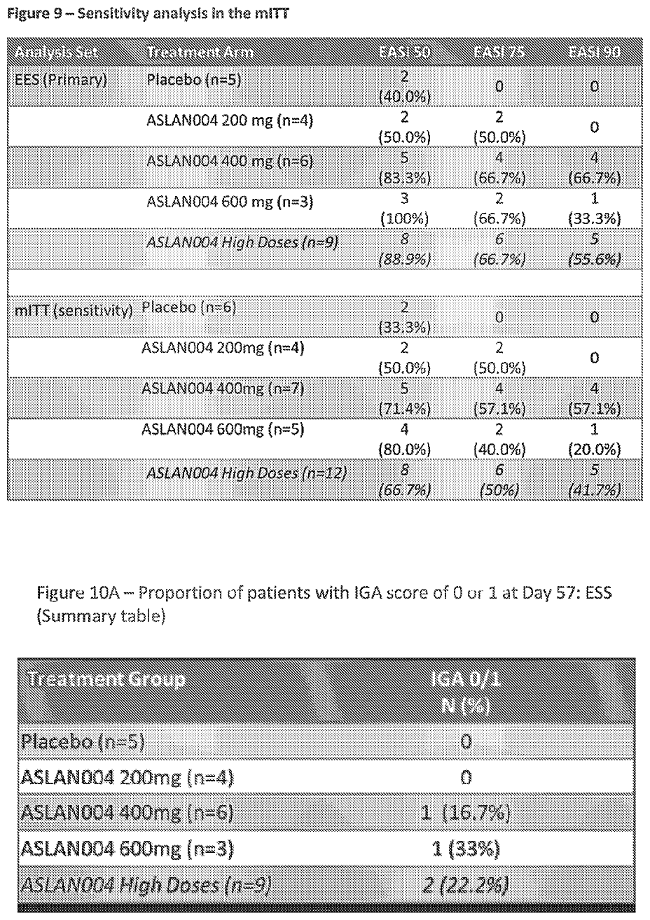

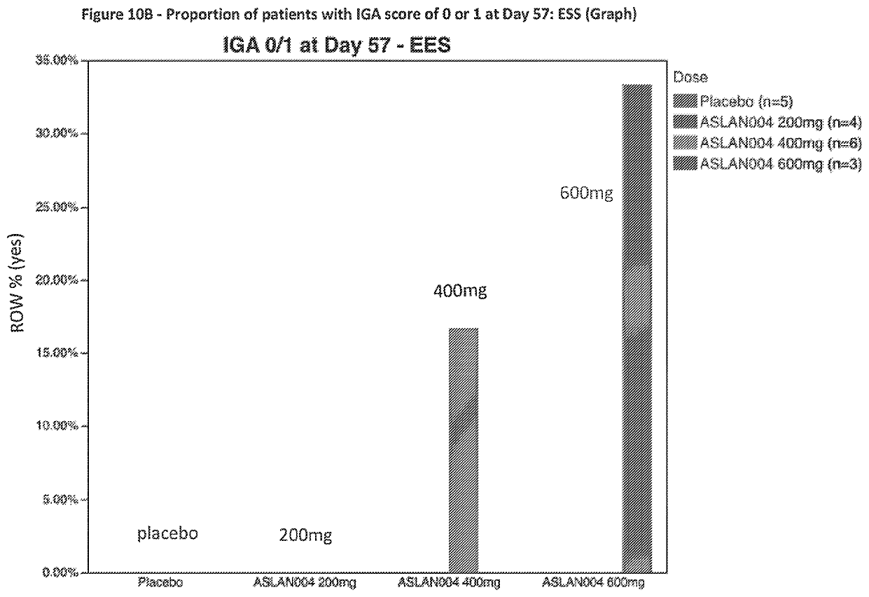

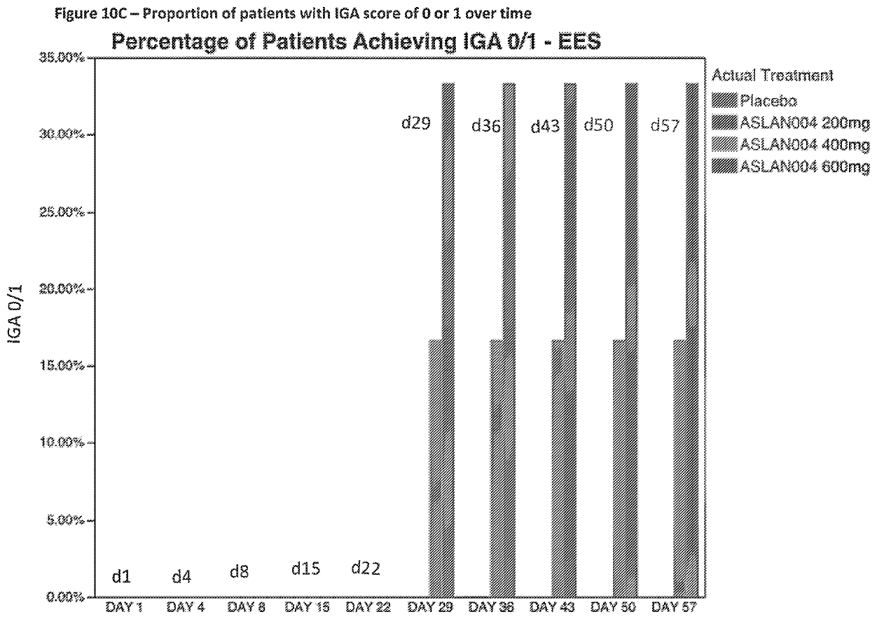

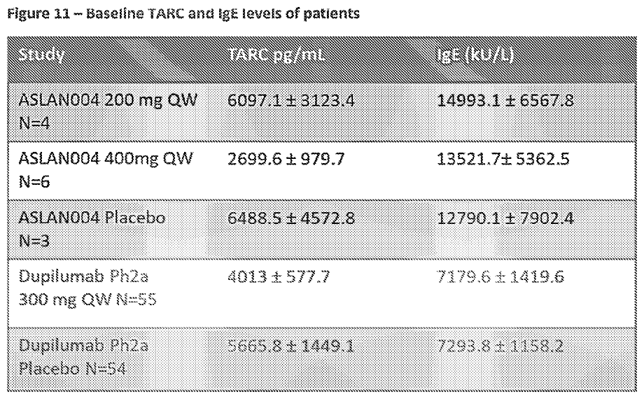

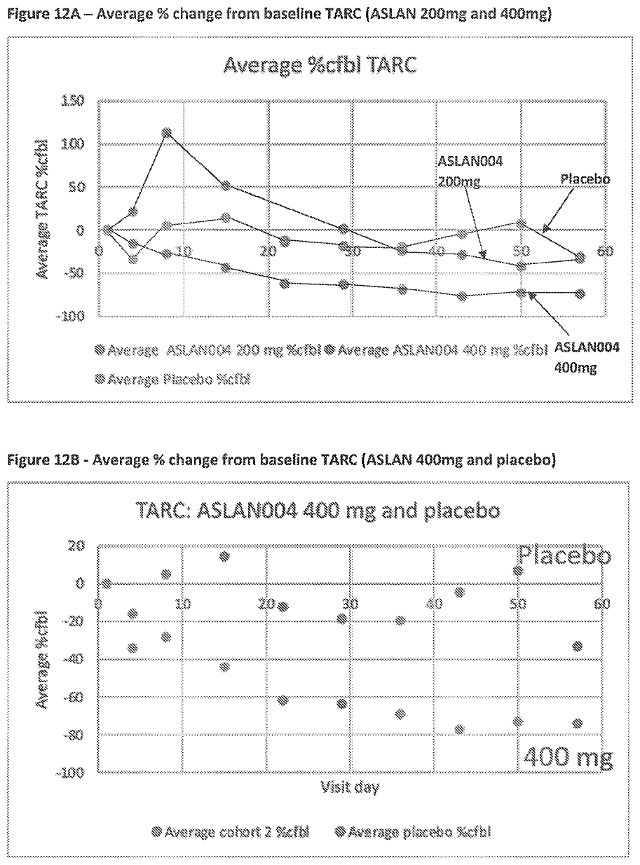

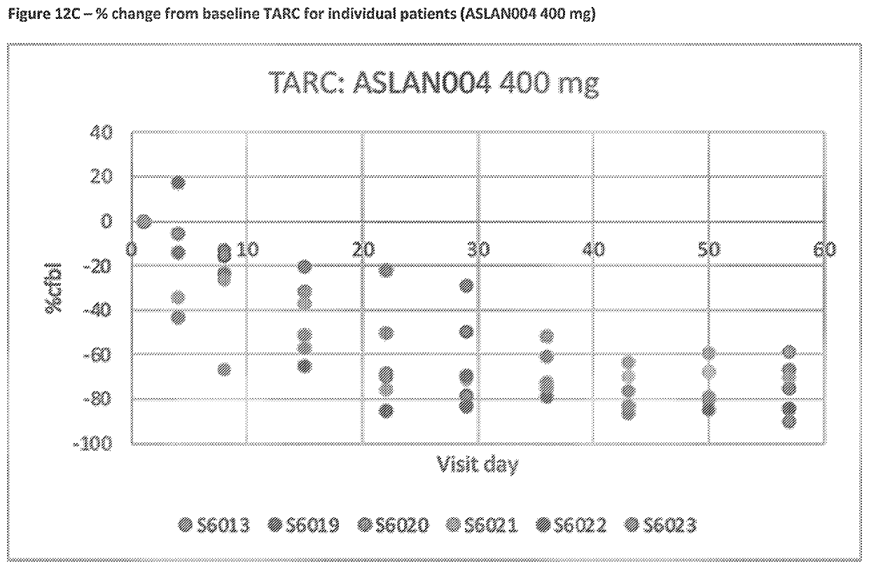

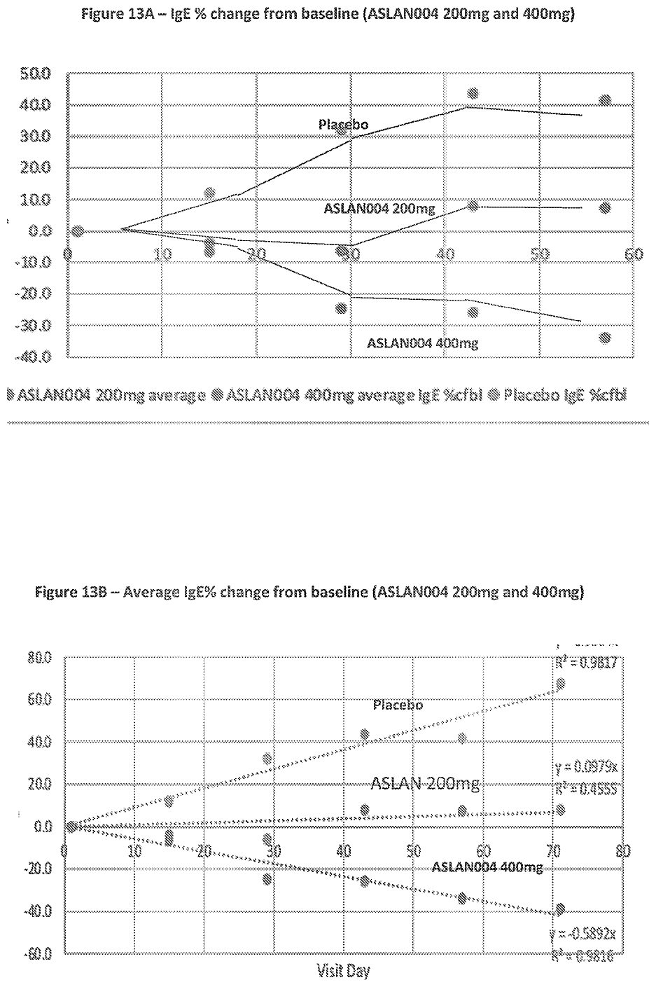

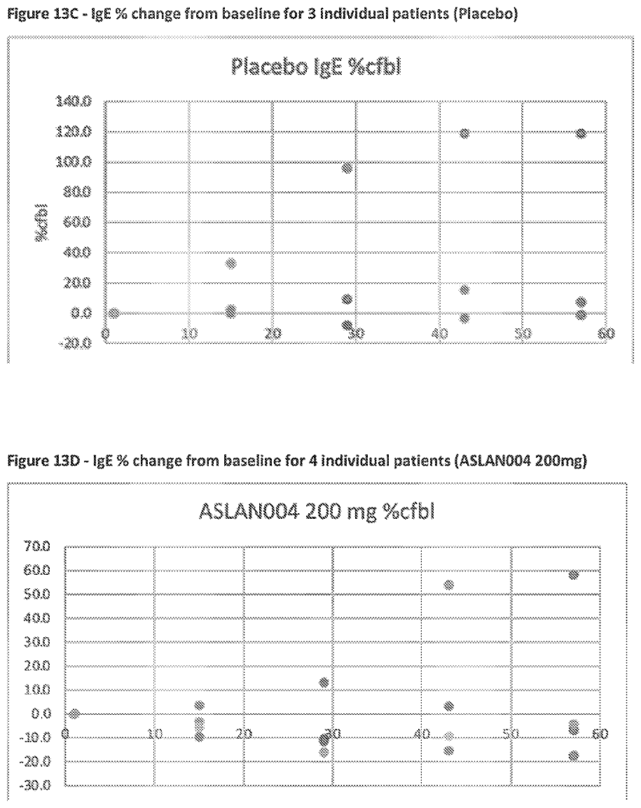

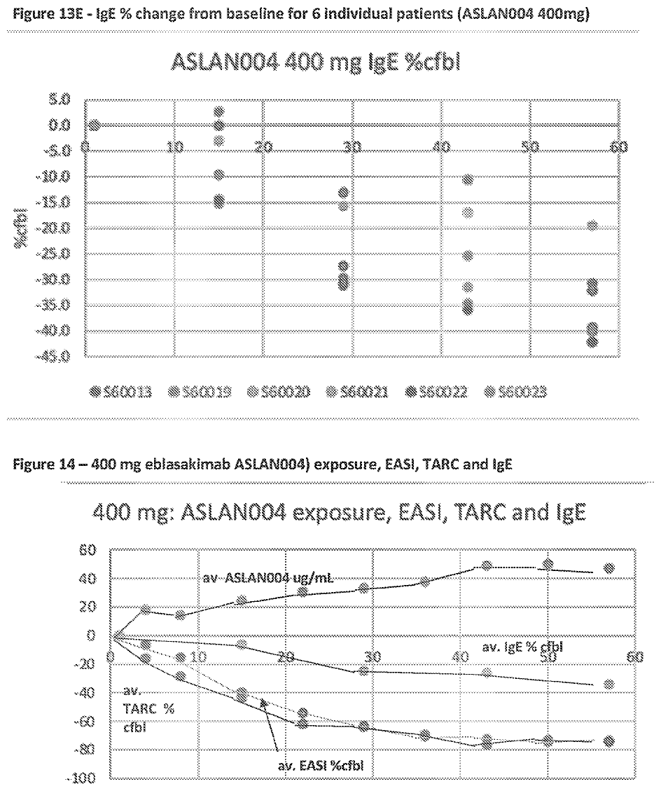

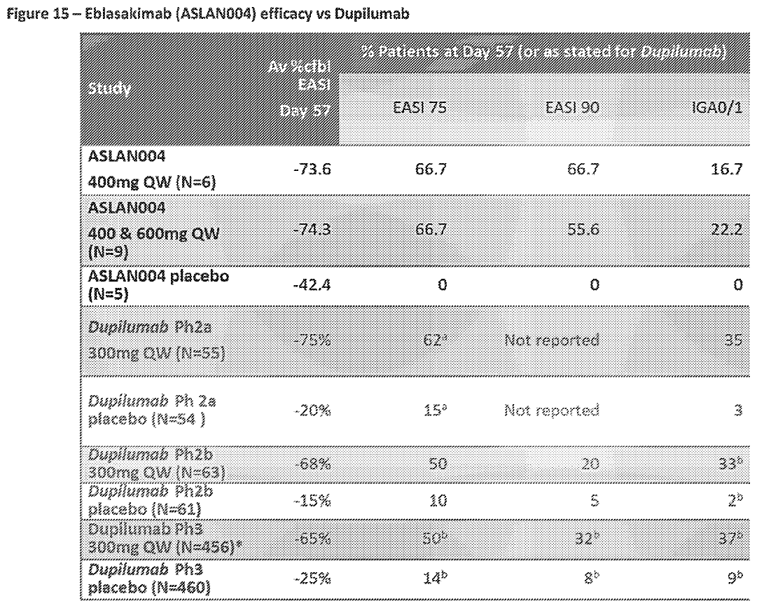

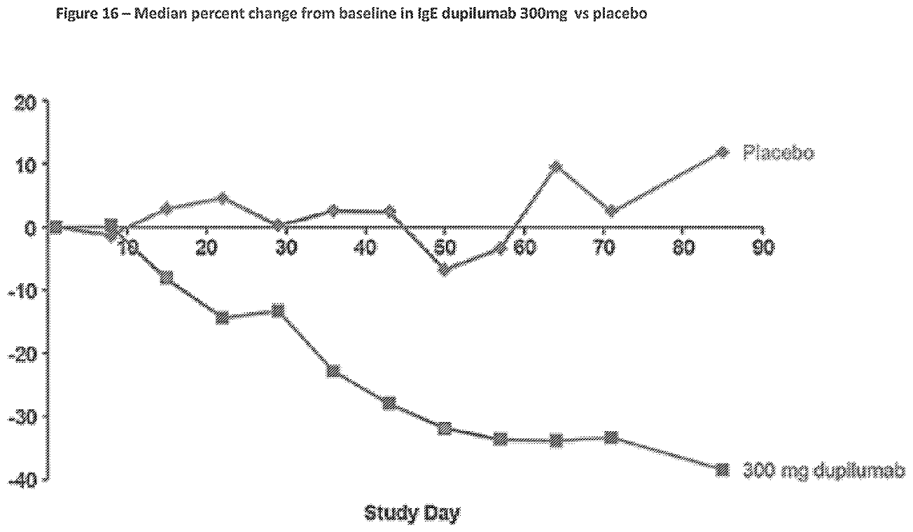

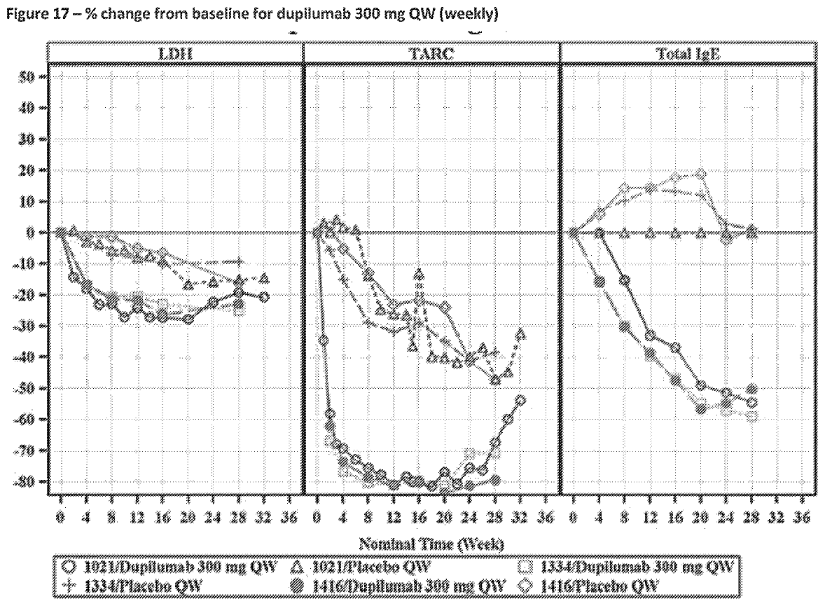

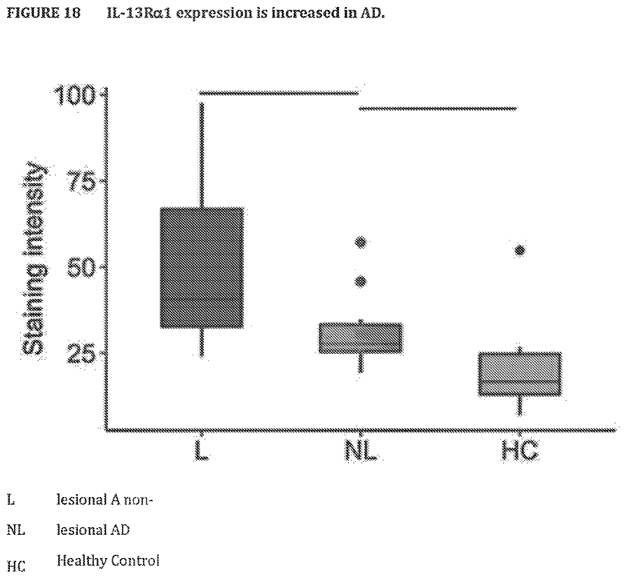

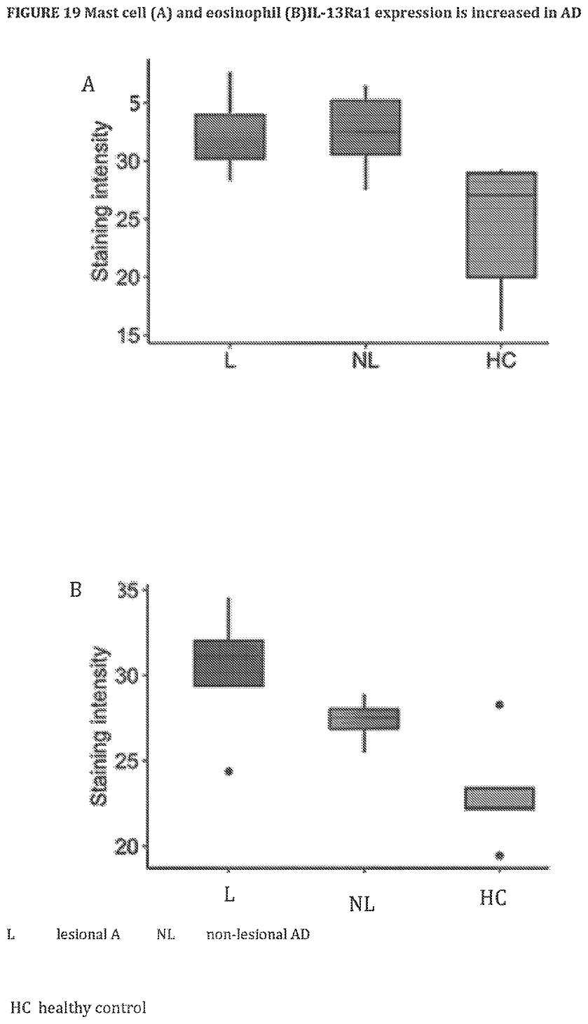

Details of patients are shown in FIG. 1. Initially the doses were given weekly (QW). Within each cohort, patients were randomized in a 3:1 ratio of EBLASAKIMAB: Placebo

### Results

Table 1 shows the % change in baseline in EASI score at Day 57 (8 weeks).

TABLE 1
% change in baseline in EASI score at Day 57 (8 weeks)

| | | | Diff (vs. |
|---|---|---|---|
| Treatment Arm | N | Mean | Placebo) |
| Placebo | 5 | −42.4% | — |
| ASLAN004 200 mg QW | 4 | −49.5% | −7.1% |
| ASLAN004 400 mg QW | 6 | −73.6% | −31.2% |
| ASLAN004 600 mg QW | 3 | −75.8% | −33.4% |
| ASLAN004 High Doses | 9 | −74.3% | −31.9% |

Table 2 shows the % change in baseline in EASI score at Day 29 (4 weeks).

TABLE 2
% change in baseline in EASI score at Day 29 (4 weeks)

| | | | Diff (vs. |
|---|---|---|---|
| Treatment Arm | N | Mean | Placebo) |
| Placebo | 5 | −30.5% | — |
| ASLAN004 200 mg | 4 | −51.2% | −20.7% |
| ASLAN004 400 mg | 6 | −63.7% | −33.2% |
| ASLAN004 600 mg | 3 | −53.6% | −23.1% |
| ASLAN004 High Doses | 9 | −60.4% | −29.8% |

Table 3 shows the sensitivity analysis in the mITT (modified intention to treat) set at Day 57

TABLE 3
Sensitivity analysis in the mITT at Day 57

| | | | | Diff (vs. |
|---|---|---|---|---|
| Analysis Set | Treatment Arm | N | Mean | Placebo) |
| EES | Placebo | 5 | −42.9% | — |
| (Primary) | ASLAN004 200 mg | 4 | −49.5% | −7.1% |
| | ASLAN004 400 mg | 6 | −73.6% | −31.2% |
| | ASLAN004 600 mg | 3 | −75.8% | −33.4% |
| mITT | Placebo | 6 | −31.4% | — |
| (sensitivity) | ASLAN004 200 mg | 4 | −49.5% | −18.9% |
| | ASLAN004 400 mg | 7 | −63.1% | −31.6% |
| | ASLAN004 600 mg | 5 | −47.1% | −15.7% |

Table 4 shows a summary of the proportion of patients achieving EASI 50, EASI 75 and EASI 90 at Day 57.

TABLE 4
EASI 50, EASI 75 and EASI 90 at Day 57

| | EASI 50 | EASI 75 | EASI 90 |
|---|---|---|---|
| Treatment Group | N (%) | N (%) | N (%) |
| Placebo (n = 5) | 2 | 0 | 0 |
| | (40.0%) | | |
| ASLAN004 200 | 2 | 2 | 0 |
| mg (n = 4) | (50.0%) | (50.0%) | |
| ASLAN004 400 | 5 | 4 | 4 |
| mg (n = 6) | (83.3%) | (66.7%) | (66.7%) |
| ASLAN004 600 | 3 | 2 | 1 |
| mg (n = 3) | (100%) | (66.7%) | (33.3%) |
| ASLAN004 High | 8 | 6 | 1 |
| Doses (n = 9) | (88.9%) | (66.7%) | (55.6%) |

The results indicate that EBLASAKIMAB results in a significant improvement in EASI score compared to placebo. In particular, at week 8, the average reduction in EASI from baseline at therapeutic doses (400 mg and 600 mg cohorts) was 74% (n=9) compared to 42% (n=5) for patients on placebo.
89% achieved EASI-50 versus 40% on placebo;
67% achieved EASI-75 versus 0% on placebo;
56% achieved EASI-90 versus 0% on placebo

The results further suggest that EBLASAKIMAB has a comparable or in some cases a higher efficacy compared to Dupilumab, thus demonstrating the potential of EBLASAKIMAB as an alternative therapy for the treatment and management of atopic dermatitis.

### Example 2

In the 32 patients that completed at least 29 days of dosing across all sites, defined in the protocol as the efficacy evaluable data set, the average reduction from baseline in EASI at 8 weeks was 73% (n=19) compared to 44% (n=13) for patients on placebo (p=0.007¹).

The proportion of patients with adverse events and treatment-related adverse events were similar across treatment and placebo arms. There were no incidences of conjunctivitis in the expansion cohort.

TABLE 5

| | RITT (n = 29) | | | ITT (n = 38) | | |
|---|---|---|---|---|---|---|
| | 600 mg | Placebo | p- | 600 mg | Placebo | p- |
| Endpoint | (n = 16) | (n = 13) | value¹ | (n = 22) | (n = 16) | value¹ |
| Mean % change from | −64.9 | −27.2 | 0.021 | −61.3 | −31.9 | 0.023 |
| baseline in EASI | | | | | | |
| EASI-50 (%) | 81.3 | 30.8 | 0.008 | 77.3 | 37.5 | 0.016 |
| EASI-75 (%) | 68.8 | 15.4 | 0.005 | 50.0 | 12.5 | 0.018 |
| EASI-90 (%) | 37.5 | 15.4 | 0.183 | 27.3 | 12.5 | 0.245 |
| IGA 0/1 (%) | 43.8 | 15.4 | 0.107 | 31.8 | 18.8 | 0.301 |
| Mean % change from | −38.6 | −15.3 | 0.051 | −37.1 | −15.7 | 0.032 |
| baseline in peak | | | | | | |
| pruritis Numerical | | | | | | |
| Rating Scale | | | | | | |
| Mean change from | −9.8 | −2.5 | 0.007 | −9.0 | −3.5 | 0.014 |
| baseline in POEM | | | | | | |

¹One-sided p-value

Eblasakimab achieved a statistically significant improvement (p<0.025) versus placebo in the primary efficacy endpoint of percent change from baseline in the Eczema Area Severity Index (EASI), and also showed significant improvements (p<0.05) in other key efficacy endpoints: EASI-50, EASI-75, peak pruritis and the Patient-Oriented Eczema Measure (POEM).

Following discussions with the Data Monitoring Committee prior to unblinding, a Revised ITT population (RITT, n=29) was defined to exclude one study site at which all patients enrolled in the study appeared atypical of moderate-to-severe AD patients based on biomarkers, such as TARC, and patient medical history. In the RITT population, which is more comparable to other published studies in moderate-to-severe AD, EBLASAKIMAB also achieved a statistically significant improvement (p<0.025) versus placebo in percent change from baseline in EASI and showed a greater improvement over placebo in the key efficacy endpoints versus the ITT population.

### Example 3

A topic dermatitis patients were enrolled in a multi-center, randomized, double-blind, placebo-controlled, multiple ascending dose study of the safety, tolerability, and pharmacokinetics of subcutaneously delivered Eblasakimab. Whilst patients with “symptoms” such as itchy skin may present in the clinic, the underlying cause of this can be a whole plethora of pathologies. The range of TARC levels presented in the clinical trial for patients with moderate to severe atopic dermatitis was from 214 pg/ml to about 22,600 pg/ml. Baseline IgE levels in patients determined to have the relevant criteria for moderate to severe atopic dermatitis ranged from 434 pg/ml to 19,175 pg/ml.

### Example 4

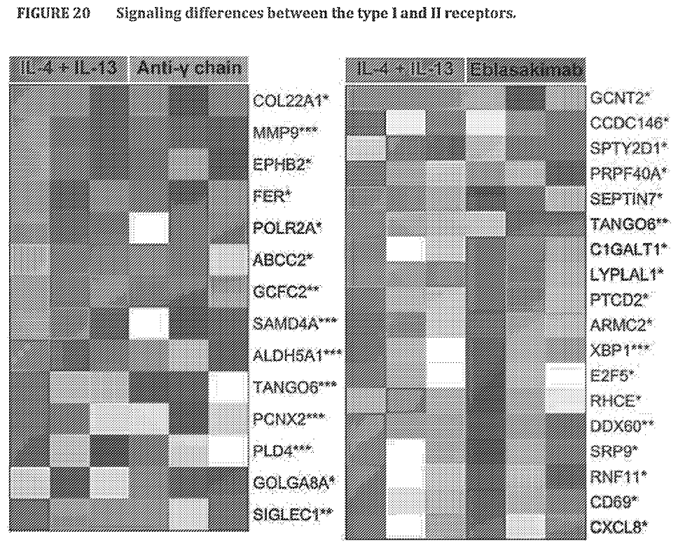

IHC was performed on lesional (L) and non-lesional (NL) skin from 14 AD patients and 10 matched controls (HC). Skin samples were stained for IL-13 Rα1, tryptase, and major basic protein to determine the distribution of IL-13 Rα1 in AD and its relation to mast cells and eosinophils. U937 cells, a monocyte line, were used to evaluate the function of the type I and II receptor as these cells express both receptors. Cells were incubated with eblasakimab (anti-IL-13 R [symbol] 1) to block type II, anti-common γ chain to block type I, and anti-IL4R [symbol] to block both type I and type II receptors. After 24 hour incubations, cells were stimulated with vehicle or a mixture of IL-4+IL-13 and subjected to RNA sequencing. IHC data were quantified using ImageJ. A differential expression analysis was conducted on RNA sequencing data using the DESeq2 package for R. IHC showed increased IL-13 Rα1 staining in L (P<0.001) and NL (P=0.045) AD skin compared to HCs. The average IL-13Rα1 staining intensity of mast cells was increased in L (P=0.034) and NL (p=0.031) AD samples compared to HCs. The average IL-13 Rα1 staining intensity of eosinophils was also increased in L (p=0.024) and NL (P=0.046) AD skin compared to HCs. Type I receptor blockade with an anti-common γ chain antibody resulted in upregulation of genes such as MMP9(P<0.001). Type II receptor blockade with eblasakimab resulted in suppression of genes such as XBP1 (P<0.001) and CXCL8 (P=0.046). FIG. 20 shows differentially expressed genes with type I receptor blockade using an anti-chain antibody. B) Differentially expressed genes with type II receptor blockade using eblasakimab.

### SUMMARY

IL-13 Rα1 expression is increased in both L and NL AD skin compared to HCs Mast cell IL-13 Rα1 expression is increased in L and NL AD skin compared to HCs Eosinophil IL-13 Rα1 expression is increased in L and NL AD skin compared to HCs There are signaling differences between the type I and II receptors

1-25 (canceled)

26. A method of treatment by administering subcutaneously an antibody or antigen binding fragment thereof, which is an inhibitor of signalling through of the IL-13 Rα1 by binding the said receptor, for moderate, severe or very severe atopic dermatitis poorly controlled by existing approved medicaments, including topical medicine, oral medicines and/or biological medicines (by parenteral administration of a treatment cycle comprising a dose in the range 300 mg to 600 mg, (such as 400 to 600 mg), wherein the disease baseline is characterised by a EASI score of 16 or above (such as 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72); and wherein the antibody or antigen binding fragment thereof comprises a VH domain comprising an amino acid sequence shown in SEQ ID NO: 51 or a sequence at least 95% identical thereto, and a VL domain comprising an amino acid sequence shown in SEQ ID NO: 53 or a sequence at least 95% identical thereto.

27. A method according to claim 26, wherein the atopic dermatitis is lesional.

28. A method according to claim 26, wherein the antibody or fragment is administered to a patient who has previously received dupilumab.

29. A method according to claim 26, wherein the IL-13 Rα1 has reduced side effects and/or improved efficacy in comparison to dupilumab.

30. A method according to claim 26, wherein the base line TARC levels are at least 1,115 pg/ml, for example in the range 1,115 pg/ml to 4,300 pg/ml or above 4,300 pg/mL

31. A method according to claim 26, wherein the baseline STAT6 is above normal.

32. A method according to claim 26, wherein the patient has a baseline IGA score of 3 or more, which is moderate to very severe atopic dermatitis, for example 3(moderate atopic dermatitis), 4 (severe atopic dermatitis) or 5 (very severe atopic dermatitis).

33. A method according to claim 26, wherein the baseline IgE levels are at a level of at least 150 KU/L, for example 1000, 1500, 2,000, 2,500, 3,000, 3500, 4,000, 4,500,5,000, 5,500, 6,000, 6,500, 7,000, 7,500, 8,000, 8,500, 9,000, 9,50 KU/L, such as at 10,000 KU/L +/−2,000

34. A method according to claim 26, wherein the one or more baseline parameters (selected from TARC levels, STAT6 is above normal, IGA score of 3 or more and IgE levels are at a level of at least 150 KU/L have been measured prior to dosing.

35. A method according to claim 34, wherein the patient prior to starting treatment has been identified as in the desired patient population.

36. A method according to claim 26, wherein the atopic dermatitis is severe.

37. A method according to claim 26, wherein a reduction in EASI score is present after about two weeks from administration of the first dose (such as day 15).

38. A method according to claim 26, wherein the dose is 300 mg or 400 mg.

39. A method according to claim 26, wherein the dose is 600 mg.

40. A method according to claim 26, wherein 90% of the patient population has an EASI 50 at about day 57.

41. A method according to claim 26, wherein the treatment cycles comprises, a first dose at 600 mg, followed by three weekly doses of 400 mg.

42. A method of treatment by administering subcutaneously a treatment cycle comprising a dose in the range 300 mg to 600 mg, of an antibody or antigen binding fragment thereof, which is an inhibitor of signalling through of the IL-13 Rα1 by binding the said receptor, to a patient with moderate atopic dermatitis, severe or very severe atopic dermatitis, wherein the disease baseline is characterised by a EASI score of 16 or above (such as 17, 18, 19, 20, 21, 22, 23, 24, 25, 26, 27, 28, 29, 30, 31,32, 33, 34, 35, 36, 37, 38, 39, 40, 41, 42, 43, 44, 45, 46, 47, 48, 49, 50, 51, 52, 53, 54, 55,56, 57, 58, 59, 60, 61, 62, 63, 64, 65, 66, 67, 68, 69, 70, 71, 72); and wherein the antibody or antigen binding fragment thereof comprises a VH domain comprising an amino acid sequence shown in SEQ ID NO: 51 or a sequence at least 95% identical thereto, and a VL domain comprising an amino acid sequence shown in SEQ ID NO: 53or a sequence at least 95% identical thereto.


SEQUENCE LISTING (64 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDRH3
  VARIANT 1: note = X denotes Phe, Met, Gln, Leu or Val
  VARIANT 6: note = X denotes Ser or Ala
  VARIANT 7: note = X denotes Phe, Leu, Ala or Met
  VARIANT 9: note = X denotes Tyr, Gln, Lys, Arg, Trp, His, Ala, Thr, Ser, Asn or Gly
XPNWGXXDX

SEQ ID NO: 4 - protein, 9 aa, synthetic construct
  REGION 1..9: note = CDR H3 SEQUENCE
FPNWGALDQ

SEQ ID NO: 5 - protein, 9 aa, synthetic construct
  REGION 1..9: note = CDR H3 SEQUENCE
VPNMGSLDT

SEQ ID NO: 6 - protein, 9 aa, synthetic construct
  REGION 1..9: note = CDR H3 SEQUENCE
FPNWGSMDA

SEQ ID NO: 7 - protein, 9 aa, synthetic construct
  REGION 1..9: note = CDR H3 SEQUENCE
FPNWGSLDH

SEQ ID NO: 8 - protein, 9 aa, synthetic construct
  REGION 1..9: note = CDR H3 SEQUENCE
MPNWGSFDY

SEQ ID NO: 9 - protein, 9 aa, synthetic construct
  REGION 1..9: note = CDR H3 SEQUENCE
MPNWGSFDT

SEQ ID NO: 10 - protein, 9 aa, synthetic construct
  REGION 1..9: note = CDR H3 SEQUENCE
MPNWGSLDH

SEQ ID NO: 11 - protein, 9 aa, synthetic construct
  REGION 1..9: note = CDR H3 SEQUENCE
MPNWGSFDS

SEQ ID NO: 12 - protein, 9 aa, synthetic construct
  REGION 1..9: note = CDR H3 SEQUENCE
MPNWGSLDT

SEQ ID NO: 13 - protein, 9 aa, synthetic construct
  REGION 1..9: note = CDR H3 SEQUENCE
MPNWGSLDA

SEQ ID NO: 14 - protein, 9 aa, synthetic construct
  REGION 1..9: note = CDR H3 SEQUENCE
MPNWGSLDN

SEQ ID NO: 15 - protein, 9 aa, synthetic construct
  REGION 1..9: note = CDR H3 SEQUENCE
MPNWGALDS

SEQ ID NO: 16 - protein, 9 aa, synthetic construct
  REGION 1..9: note = CDR H3 SEQUENCE
MPNWGSLDN

SEQ ID NO: 17 - protein, 9 aa, synthetic construct
  REGION 1..9: note = CDR H3 SEQUENCE
MPNWGSLDY

SEQ ID NO: 18 - protein, 9 aa, synthetic construct
  REGION 1..9: note = CDR H3 SEQUENCE
MPNWGSFDH

SEQ ID NO: 19 - protein, 9 aa, synthetic construct
  REGION 1..9: note = CDR H3 SEQUENCE
MPNWGSLDS

SEQ ID NO: 20 - protein, 9 aa, synthetic construct
  REGION 1..9: note = CDR H3 SEQUENCE
MPNWGSLDG

SEQ ID NO: 21 - protein, 9 aa, synthetic construct
  REGION 1..9: note = CDR H3 SEQUENCE
VPNWGSLDG

SEQ ID NO: 22 - protein, 28 aa, synthetic construct
  REGION 1..28: note = CDR H3 SEQUENCE
CARFPNWGSLDHWGQGTLVTVSSASIKG

SEQ ID NO: 23 - protein, 28 aa, synthetic construct
  REGION 1..28: note = CDR H3 SEQUENCE
CARMPNWGSLDHWGQGTLVTVSSASTKG

SEQ ID NO: 24 - protein, 28 aa, synthetic construct
  REGION 1..28: note = CDR H3 SEQUENCE
CARMPNWGSFDYWGQGTLVTVSSASIKG

SEQ ID NO: 25 - protein, 12 aa, synthetic construct
  REGION 1..12: note = CDR H3 SEQUENCE
VRMPNWGSLDHW

SEQ ID NO: 26 - protein, 27 aa, synthetic construct
  REGION 1..27: note = CDR H3 SEQUENCE
VRMPNWGSLDHWGQGTLVTVSSADIKG

SEQ ID NO: 27 - protein, 27 aa, synthetic construct
  REGION 1..27: note = CDR H3 SEQUENCE
ARMPNWGSLDHWGQGTLVTVSSASIKG

SEQ ID NO: 28 - protein, 25 aa, synthetic construct
  REGION 1..25: note = CDR H3 SEQUENCE
FPNWGSFDYWGQGTLVTVSSASIKG

SEQ ID NO: 29 - protein, 9 aa, synthetic construct
  REGION 1..9: note = CDR H3 SEQUENCE
VPNWGSLDA

SEQ ID NO: 30 - protein, 9 aa, synthetic construct
  REGION 1..9: note = CDR H3 SEQUENCE
FPNWGSFDY

SEQ ID NO: 31 - protein, 12 aa, synthetic construct
  REGION 1..12: note = CDRL1
RASQSISSSYLA

SEQ ID NO: 32 - protein, 7 aa, synthetic construct
  REGION 1..7: note = CDRL2
GASSRAT

SEQ ID NO: 33 - skipped

SEQ ID NO: 34 - protein, 5 aa, synthetic construct
  REGION 1..5: note = CDRL3 SEQUENCE
QRYAT

SEQ ID NO: 35 - protein, 5 aa, synthetic construct
  REGION 1..5: note = CDRL3 SEQUENCE
QRYST

SEQ ID NO: 36 - protein, 5 aa, synthetic construct
  REGION 1..5: note = CDRL3 SEQUENCE
QMYST

SEQ ID NO: 37 - protein, 5 aa, synthetic construct
  REGION 1..5: note = CDRL3 SEQUENCE
QQVGT

SEQ ID NO: 38 - protein, 5 aa, synthetic construct
  REGION 1..5: note = CDRL3 SEQUENCE
QQVST

SEQ ID NO: 39 - protein, 5 aa, synthetic construct
  REGION 1..5: note = CDRL3 SEQUENCE
QQYST

SEQ ID NO: 40 - protein, 5 aa, synthetic construct
  REGION 1..5: note = CDRL3 SEQUENCE
QSYST

SEQ ID NO: 41 - protein, 5 aa, synthetic construct
  REGION 1..5: note = CDRL3 SEQUENCE
QQYAT

SEQ ID NO: 42 - protein, 5 aa, synthetic construct
  REGION 1..5: note = CDRL3 SEQUENCE
QQYSS

SEQ ID NO: 43 - protein, 5 aa, synthetic construct
  REGION 1..5: note = CDRL3 SEQUENCE
QTYST

SEQ ID NO: 44 - protein, 5 aa, synthetic construct
  REGION 1..5: note = CDRL3 SEQUENCE
QQYGS

SEQ ID NO: 45 - protein, 5 aa, synthetic construct
  REGION 1..5: note = CDRL3 SEQUENCE
QQYAS

SEQ ID NO: 46 - protein, 5 aa, synthetic construct
  REGION 1..5: note = CDRL3 SEQUENCE
QQYEA

SEQ ID NO: 47 - protein, 5 aa, synthetic construct
  REGION 1..5: note = CDRL3 SEQUENCE
QQYET

SEQ ID NO: 48 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - skipped

SEQ ID NO: 56 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 62 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC